(19) United States
(12) Patent Application Publication
(10) Pub. No.: US 2024/0382110 A1
(43) Pub. Date: Nov. 21, 2024

(54) DATA GENERATION DEVICE, LEARNING SYSTEM, ESTIMATION SYSTEM, DATA GENERATION METHOD, AND RECORDING MEDIUM

(71) Applicant: NEC Corporation, Minato-ku, Tokyo (JP)

(72) Inventors: Chenhui HUANG, Tokyo (JP); Yoshitaka Nozaki, Tokyo (JP); Kenichiro Fukushi, Tokyo (JP)

(73) Assignee: NEC Corporation, Minato-ku, Tokyo (JP)

(21) Appl. No.: 18/691,486

(22) PCT Filed: Oct. 19, 2021
(86) PCT No.: PCT/JP2021/038615

§ 371 Date: Mar. 13, 2024

(51) Int. Cl. A61B 5/11; A61B 5/00; G16H 20/30; G16H 50/20; G16H 50/30

(52) U.S. Cl. CPC A61B 5/112; A61B 5/6807; A61B 5/742; G16H 20/30; G16H 50/20; G16H 50/30; A61B 2560/045; A61B 2560/0462; A61B 2562/0219

(57) ABSTRACT

Provided is a data generation device that acquires pair data constituted by a combination of measurement gait data relating to sensor data measured in accordance with the movement of a user's feet and a response variable corresponding to the measurement gait data, generates a measurement dataset vector by combining the response variable and a feature amount vector calculated using a feature amount extracted from measurement gait data, generates a covariance matrix relating to a plurality of pair data, generates pseudo gait data using the measurement gait data, generate a pseudo dataset vector by combining a pseudo feature amount vector calculated using a pseudo feature amount extracted from the pseudo gait data, and a pseudo response variable generated using a covariance matrix relating to the pseudo feature amount vector, and outputs the dataset.

### TECHNICAL FIELD

The present disclosure relates to a data generation device that generates a dataset used for learning of a model for estimating a physical condition using gait data, and the like.

### BACKGROUND ART

With increasing interest in healthcare, attention has been focused on a service for providing information relevant to features (also referred to as gait) included in a walking pattern. For example, a technique for analyzing a gait based on sensor data measured by a sensor mounted on footwear such as shoes has been developed. Features of gait events associated with health status appear in time-series sensor data.

PTL 1 discloses a learning device that generates a learning model using rehabilitation data collected within a predetermined period. The device of PTL 1 generates rehabilitation data including an index indicating a degree of recovery of a trainee and a setting parameter as learning data. The device of PTL 1 performs machine learning using learning data to generate a learning model that outputs a recommended value of a setting parameter using an index as an input.

NPL 1 discloses extension of inertial sensor-based walking data. In the method of NPL 1, walking data is extended by applying random temporal fluctuations to time-series sensor data measured by an inertial sensor.

### CITATION LIST

### Patent Literature

PTL 1: JP 2021-007481 A

### Non Patent Literature

NPL 1: L. Tran and D. Choi, “Data Augmentation for Inertial Sensor-Based Gait Deep Neural Network”, IEEE Access, Vol. 8, pp. 12364-12378 (2020).

### SUMMARY OF INVENTION

### Technical Problem

In the method of PTL 1, it is necessary to collect as much rehabilitation data as possible in order to generate a learning model with sufficient accuracy. However, in order to collect a sufficient number of pieces of rehabilitation data, it is necessary to increase walking training of the trainee, and enormous time and effort are required for collecting data.

In the method of NPL 1, the time-series sensor data relevant to the explanatory variable is extended, and the response variable is associated with the extended explanatory variable, thereby extending the dataset used for learning. That is, according to the method of NPL 1, it is possible to save time and labor required for collecting data. For example, according to the method of NPL 1, when the response variable is a fixed value like a personal authentication identifier (ID), the dataset can be extended. However, in the method of PTL 1, when the response variable is a continuous value, the response variable cannot be associated with the extended explanatory variable, and thus the dataset cannot be extended.

An object of the present disclosure is to provide a data generation device and the like capable of generating a dataset used for learning even when a response variable is a continuous value.

### Solution to Problem

A data generation device according to an aspect of the present disclosure includes an acquisition unit that acquires pair data by combing measurement gait data relating to sensor data measured in accordance with the movement of a user's feet and a response variable relevant to the measurement gait data, a measurement data processing unit that generates a measurement dataset vector by combining the response variable and a feature amount vector calculated using a feature amount extracted from the measurement gait data, and generates a covariance matrix relating to a plurality of pieces of the pair data, a pseudo data generation unit that generates pseudo gait data using the measurement gait data, and generates a pseudo dataset vector by combining a pseudo feature amount vector calculated using a pseudo feature amount extracted from the pseudo gait data and a pseudo response variable generated using a covariance matrix relating to the pseudo feature amount vector, and an output unit that outputs the dataset including the measurement dataset vector and the pseudo dataset vector.

A data generation method according to an aspect of the present disclosure includes acquiring pair data in which measurement gait data relating to sensor data measured in accordance with a movement of a user's feet and a response variable relevant to the measurement gait data are combined, generating a measurement dataset vector by combining the response variable and a feature amount vector calculated using a feature amount extracted from the measurement gait data, generating a covariance matrix for a plurality of four pieces of the pair data, generating pseudo gait data using the measurement gait data, generating a pseudo dataset vector by combining a pseudo feature amount vector calculated using a pseudo feature amount extracted from the pseudo gait data and a pseudo response variable generated using the covariance matrix relating to the pseudo feature amount vector, and outputting a dataset including the measurement dataset vector and the pseudo dataset vector.

A program according to an aspect of the present disclosure causes a computer to execute processing of acquiring pair data by combing measurement gait data relating to sensor data measured in accordance with the movement of a user's feet and a response variable relevant to the measurement gait data, processing of generating a measurement dataset vector by combining the response variable and a feature amount vector calculated using a feature amount extracted from the measurement gait data, processing of generating a covariance matrix relating to a plurality of pieces of the pair data, processing of generating pseudo gait data using the measurement gait data, processing of generating a pseudo dataset vector by combining a pseudo feature amount vector calculated using a pseudo feature amount extracted from the pseudo gait data and a pseudo response variable generated using the covariance matrix relating to the pseudo feature amount vector, and processing of outputting a dataset including the measurement dataset vector and the pseudo dataset vector.

### Advantageous Effects of Invention

According to the present disclosure, it is possible to provide a data generation device and the like capable of generating a dataset used for learning even when a response variable is a continuous value.

### BRIEF DESCRIPTION OF DRAWINGS

FIG. 1 is a block diagram illustrating an example of a configuration of a learning system according to a first example embodiment.

FIG. 2 is a conceptual diagram for explaining an arrangement example of a measurement device that measures sensor data used by a data generation device included in the learning system according to the first example embodiment.

FIG. 3 is a conceptual diagram for explaining an example of a coordinate system set in the measurement device that measures sensor data used by the data generation device included in the learning system according to the first example embodiment.

FIG. 4 is a conceptual diagram for explaining an example of a gait event detected from time-series sensor data used by the data generation device included in the learning system according to the first example embodiment.

FIG. 5 is a conceptual diagram for explaining an example of extraction of a feature amount by the data generation device included in the learning system according to the first example embodiment.

FIG. 6 is a conceptual diagram for explaining an example of generating a feature amount vector by the data generation device included in the learning system according to the first example embodiment.

FIG. 7 is a conceptual diagram for explaining an example of data processing by the data generation device included in the learning system according to the first example embodiment.

FIG. 8 is a conceptual diagram for explaining an example of data processing by the data generation device included in the learning system according to the first example embodiment.

FIG. 9 is a conceptual diagram for explaining an example of data processing by the data generation device included in the learning system according to the first example embodiment.

FIG. 10 is a conceptual diagram for explaining an example of data processing by the data generation device included in the learning system according to the first example embodiment.

FIG. 11 is a conceptual diagram for explaining an example of data processing by the data generation device included in the learning system according to the first example embodiment.

FIG. 12 is a conceptual diagram for explaining an example of data processing by the data generation device included in the learning system according to the first example embodiment.

FIG. 13 is a conceptual diagram for explaining an example of data processing by the data generation device included in the learning system according to the first example embodiment.

FIG. 14 is a conceptual diagram for explaining an example of data processing by the data generation device included in the learning system according to the first example embodiment.

FIG. 15 is a conceptual diagram for explaining an example of a configuration of a learning device included in the learning system according to the first example embodiment.

FIG. 16 is a flowchart for explaining an example of the operation of the data generation device included in the learning system according to the first example embodiment.

FIG. 17 is a flowchart for explaining an example of measurement data processing by the data generation device included in the learning system according to the first example embodiment.

FIG. 18 is a flowchart for explaining an example of pseudo data generation processing by the data generation device included in the learning system according to the first example embodiment.

FIG. 19 is a flowchart for explaining an example of the operation of the learning device included in the learning system according to the first example embodiment.

FIG. 20 is a graph for explaining evaluation of accuracy of a response variable estimated by an estimation model generated using a dataset not extended by a data processing device included in the learning system according to the first example embodiment.

FIG. 21 is a graph for explaining evaluation of accuracy of a response variable estimated by an estimation model generated using a dataset extended by the learning device included in the learning system according to the first example embodiment.

FIG. 22 is a graph for explaining evaluation of versatility of a response variable estimated by an estimation model generated using a dataset not extended by the data processing device included in the learning system according to the first example embodiment.

FIG. 23 is a graph for explaining evaluation of versatility of a response variable estimated by an estimation model generated using a dataset extended by the data processing device included in the learning system according to the first example embodiment.

FIG. 24 is a conceptual diagram for explaining Application Example 1-1 of the first example embodiment.

FIG. 25 is a conceptual diagram for explaining Application Example 1-1 of the first example embodiment.

FIG. 26 is a conceptual diagram for explaining Application Example 1-2 of the first example embodiment.

FIG. 27 is a block diagram illustrating an example of a configuration of an estimation system according to a second example embodiment.

FIG. 28 is a block diagram illustrating an example of a configuration of a measurement device included in the estimation system according to the second example embodiment.

FIG. 29 is a conceptual diagram for explaining an arrangement example of the measurement device included in the estimation system according to the second example embodiment.

FIG. 30 is a block diagram illustrating an example of a configuration of an estimation device included in the estimation system according to the second example embodiment.

FIG. 31 is a conceptual diagram for explaining an example of estimation by the estimation device included in the estimation system according to the second example embodiment.

FIG. 32 is a flowchart for explaining an example of the operation of the measurement device included in the estimation system according to the second example embodiment.

FIG. 33 is a flowchart for explaining an example of sensor data measurement processing by the measurement device included in the estimation system according to the second example embodiment.

FIG. 34 is a flowchart for explaining an example of gait parameter calculation processing by the measurement device included in the estimation system according to the second example embodiment.

FIG. 35 is a flowchart for explaining an example of the operation of the estimation device included in the estimation system according to the second example embodiment.

FIG. 36 is a conceptual diagram for explaining Application Example 2-1 of the second example embodiment.

FIG. 37 is a conceptual diagram for explaining Application Example 2-2 of the second example embodiment.

FIG. 38 is a conceptual diagram for explaining Application Example 2-2 of the second example embodiment.

FIG. 39 is a conceptual diagram illustrating an example of a configuration of a data generation device according to a third example embodiment.

FIG. 40 is a block diagram illustrating an example of a hardware configuration that executes control and processing according to each example embodiment.

### EXAMPLE EMBODIMENT

Hereinafter, example embodiments of the present invention will be described with reference to the drawings. However, the example embodiments described below may be technically limited for carrying out the present invention, but the scope of the invention is not limited to the following. In all the drawings used in the following description of the example embodiment, the same reference numerals are given to the same parts unless there is a particular reason. In the following example embodiments, repeated description of similar configurations and operations may be omitted.

### First Example Embodiment

Next, a learning system according to a first example embodiment will be described with reference to the drawings. The learning system according to the present example embodiment generates a learning model that outputs a response variable relating to a gait event or a physical condition of a user in accordance with an input of a feature amount generated based on a physical amount measured in accordance with walking of the user.

### (Configuration)

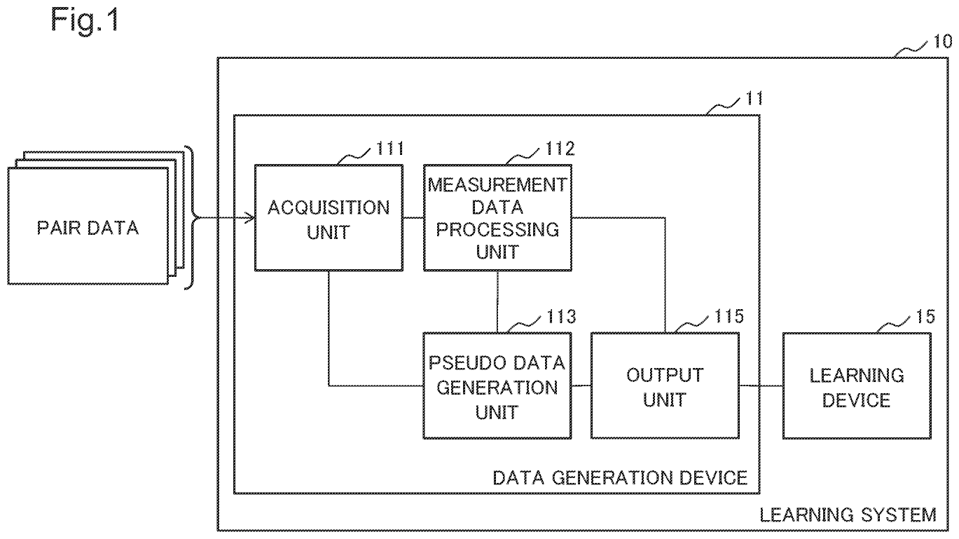

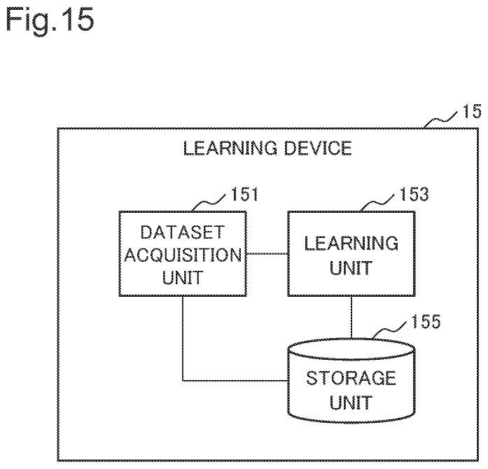

FIG. 1 is a block diagram illustrating an example of a configuration of a learning system 10 according to the present example embodiment. The learning system 10 includes a data generation device 11 and a learning device 15. FIG. 1 illustrates a detailed configuration of the data generation device 11. A detailed configuration of the learning device 15 will be described later (FIG. 15). Hereinafter, the data generation device 11 and the learning device 15 will be described in order.

### [Data Generation Device]

The data generation device 11 of the learning system 10 will be described. The data generation device 11 includes an acquisition unit 111, a measurement data processing unit 112, a pseudo data generation unit 113, and an output unit 115.

The acquisition unit 111 acquires pair data relevant to walking of the user. The pair data is data in which gait data relating to a feature (also referred to as a gait) included in the walking pattern of the user and a gait event index (also referred to as a response variable) relevant to the gait data are paired. That is, the pair data is data obtained by combining the gait data and the response variable relevant to the gait data. For example, the acquisition unit 111 acquires pair data accumulated in a database constructed in a cloud or a server (not illustrated).

The gait data is data relating to a physical amount (also referred to as sensor data) relating to the movement of the feet. For example, the gait data is time-series data of a physical amount (also referred to as sensor data) relating to the movement of the feet. For example, the gait data is a value of a gait parameter relating to an event (also referred to as a gait event) detected from the time-series sensor data. Hereinafter, the gait data based on the measured sensor data is also referred to as measurement gait data.

The gait event index (response variable) is a value relevant to the gait data. For example, the response variable is a numerical value relating to a gait parameter such as a walking speed, a stride length, a grounding angle, a separation angle, a maximum foot-raising height (sensor position), a division (traveling direction trajectory), and a toe direction of the user. For example, the response variable is a numerical value relating to the physical condition of the user estimated based on the gait parameter. For example, the response variable is a numerical value indicating the degree of pronation/supination or hallux valgus of the user's feet, left-right symmetry, or the like. For example, the response variable may be an identification number for identifying the user or a value relating to an attribute.

The time-series sensor data (also referred to as a gait waveform) for one gait cycle is a set of sensor data such as acceleration, angular velocity, and plantar angle. Hereinafter, a unit section of one gait cycle is referred to as a “gait phase”. For example, when the gait waveform for one gait cycle is equally divided into 0 to 100%, the gait phase is set every 1%. The division reference of the gait waveform for one gait cycle is not particularly limited. For example, the gait waveform for one gait cycle may be divided in accordance with the measurement condition of the sensor data or the gait parameter of the measurement target.

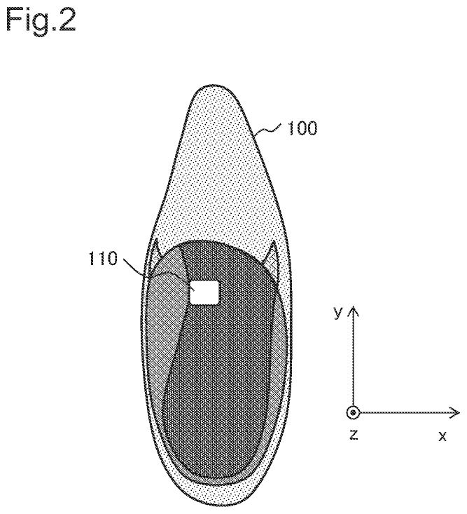

FIG. 2 is a conceptual diagram for explaining an arrangement example of a measurement device 110 that measures a physical amount relating to the movement of the feet. The measurement device 110 is disposed in a shoe 100. FIG. 2 is an example in which the measurement device 110 is installed at a position relevant to the back side of the arch of foot. For example, the measurement device 110 is disposed in an insole inserted into the shoe 100. For example, the measurement device 110 is disposed on the bottom surface of the shoe 100. For example, the measurement device 110 is embedded in the main body of the shoe 100. The measurement device 110 may be detachable from the shoe 100 or may not be detachable from the shoe 100. The measurement device 110 may be installed at a position other than the back side of the arch of the foot as long as the sensor data relating to the movement of the feet can be acquired. The measurement device 110 may be installed on a sock worn by the user or a decorative article such as an anklet worn by the user. The measurement device 110 may be directly attached to the foot or may be embedded in the foot. FIG. 2 illustrates an example in which the measurement device 110 are installed on the shoe 100 of the right foot. The measurement device 110 may be installed in the shoe 100 of the left foot. The measurement device 110 may be installed on the shoes 100 for both feet.

The measurement device 110 includes a sensor (not illustrated) that measures a physical amount relating to the movement of the feet of a user wearing footwear. The physical amount relating to the movement of the feet includes acceleration in three axis directions (also referred to as spatial acceleration) and angular velocity around three axes (also referred to as spatial angular velocity). The physical amount relating to the movement of the feet includes a speed, an angle, and a position (trajectory) calculated by integrating the acceleration and the angular velocity.

The acquisition unit 111 acquires pair data relating to the sensor data measured by the measurement device 110. The acquisition destination of the pair data is not particularly limited. For example, the acquisition unit 111 acquires pair data accumulated in a cloud, a server, or the like. For example, the acquisition unit 111 may acquire pair data stored in a mobile terminal carried by the user. The acquisition unit 111 may acquire measurement data from the measurement device 110 in real time.

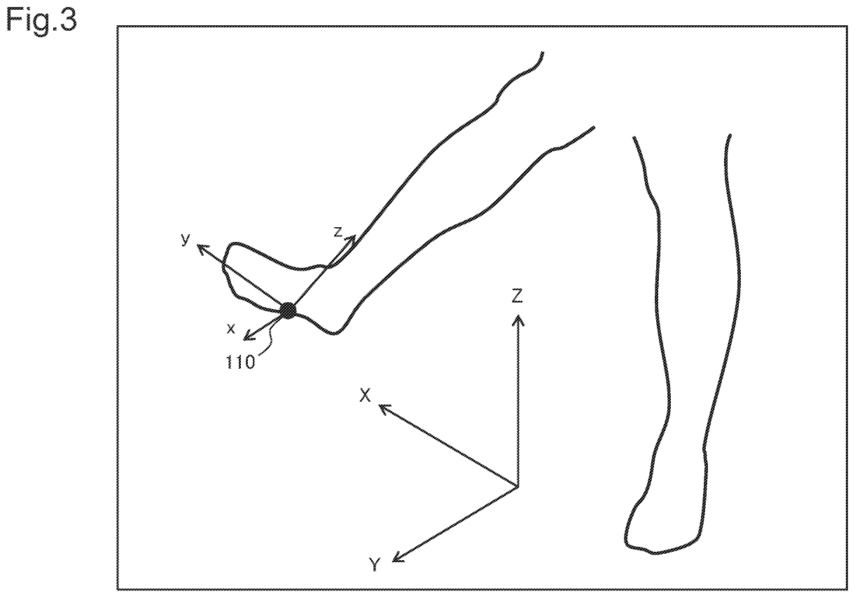

FIG. 3 is a conceptual diagram for explaining a local coordinate system (x axis, y axis, z axis) set in the measurement device 110 in a case where the measurement device 110 is installed on the back side of the arch of foot. A local coordinate system including an x direction, a y direction, and a z direction is set in the measurement device 110. A world coordinate system (X axis, Y axis, Z axis) is set for the ground. In the world coordinate system (X axis, Y axis, Z axis), in a state where the user is standing upright, a lateral direction of the user is set to an X-axis direction (rightward direction is positive), a front direction of the user (traveling direction) is set to a Y-axis direction (forward direction is positive), and a gravity direction is set to a Z-axis direction (vertically upward direction is positive). The local coordinate system set in the measurement device 110 is not limited to the example of FIG. 3. The local coordinate system can be arbitrarily set for the measurement device 110.

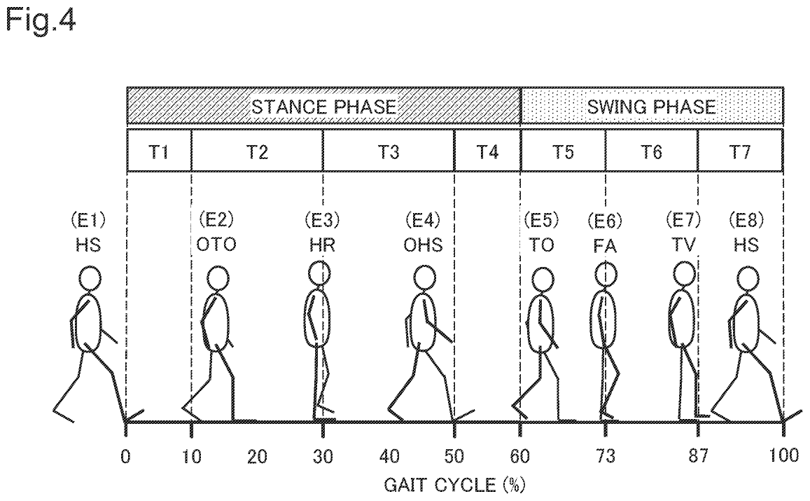

FIG. 4 is a conceptual diagram for explaining a gait event detected in one gait cycle with the right foot as a reference. The horizontal axis of FIG. 4 is a gait cycle normalized with one gait cycle of the right foot as 100%, with a time point at which the heel of the right foot lands on the ground as a start point and a time point at which the heel of the right foot next lands on the ground as an end point. The one gait cycle of one foot is roughly divided into a stance phase in which at least a part of the back side of the foot is in contact with the ground and a swing phase in which the back side of the foot is separated from the ground. In the example of FIG. 4, normalization is made such that the stance phase occupies 60% and the swing phase occupies 40%. The stance phase is further subdivided into an initial stance period T1, a mid-stance period T2, a terminal stance period T3, and a pre-swing period T4. The swing phase is further subdivided into an initial swing period T5, a mid-swing period T6, and a terminal swing period T7. The gait waveform in one gait cycle may not have a time point when the heel lands on the ground as the start point. For example, the start point of the gait waveform in one gait cycle may be set at a central time point of the stance phase.

In FIG. 4, a gait event E1 represents an event in which the heel of the right foot strikes the ground (HS: heel strike). A gait event E2 represents an event (opposite toe off) in which the toe of the left foot is separated from the ground while the sole of the right foot is in contact with the ground (OTO: Opposite Toe Off). A gait event E3 represents an event (heel rise) in which the heel of the right foot rises while the sole of the right foot is in contact with the ground (HR: Heel Rise). A gait event E4 is an event (opposite heel strike) in which the heel of the left foot strikes the ground (OHS: Opposite Heel Strike). A gait event E5 represents an event (toe off) in which the toe of the right foot is separated from the ground while the sole of the left foot is in contact with the ground (TO: Toe Off). A gait event E6 represents an event (foot adjacent) in which the left foot and the right foot cross each other while the sole of the left foot is in contact with the ground (FA: Foot Adjacent). A gait event E7 represents an event (tibia vertical) that the tibia of the right foot is approximately perpendicular to the ground with the sole of the left foot in contact with the ground (TV: Tibia Vertical). A gait event E8 represents an event (heel strike) in which the heel of the right foot touches the ground (HS: heel strike). The gait event E8 is relevant to the end point of the gait cycle starting from the gait event E1 and is relevant to the start point of the next gait cycle.

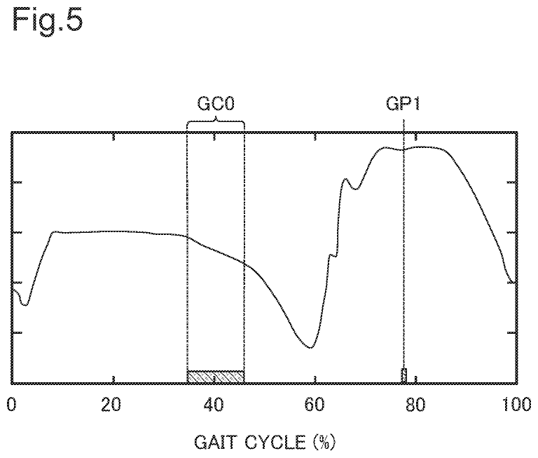

FIG. 5 is a conceptual diagram for explaining a feature amount extracted from a gait waveform. The graph of FIG. 5 is an example of a gait waveform of the plantar angle around the x axis. The gait waveform in FIG. 5 is a waveform for one gait cycle (0 to 100%) starting from the heel strike. In the example of FIG. 5, the feature amount is extracted from each of the continuous gait phases having a gait cycle of around 40%. The plurality of feature amounts extracted from the temporally continuous gait phase are integrated as a cluster (also referred to as a gait phase cluster). In the example of FIG. 5, a gait phase cluster GC0 in which continuous gait phases having a gait cycle of around 40% are integrated is set. From the gait phase cluster, a feature amount relevant to the feature amount extracted from the gait phase constituting the gait phase cluster is extracted. In the example of FIG. 5, the feature amount is extracted from a single gait phase GPI before the gait cycle of 80%. The feature amount extracted from the sensor data for one gait cycle is integrated as a feature amount vector of the one gait cycle. For example, a plurality of feature amounts extracted for each of nine types of gait waveforms relating to acceleration in three axis directions and angular velocity/angle around three axes is integrated as a feature amount vector for one gait cycle.

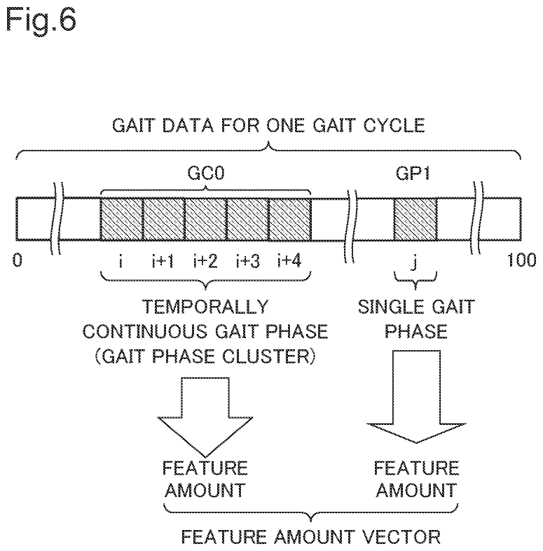

FIG. 6 is a conceptual diagram for explaining an example of a feature amount vector extracted from gait data for one gait cycle. From the gait waveform, a feature according to the gait event index related to the physical condition is extracted. The gait event index is an evaluation index of a gait event and is relevant to a response variable.

For example, the gait event index is a numerical value relating to a gait parameter of the user. For example, the gait event index is a numerical value (also referred to as a score) relating to evaluation of the gait of the user. For example, the gait event index includes the degree of pronation/supination of the foot, the degree of progression of hallux valgus, the degree of progression of knee arthropathy, the degree of physical condition such as muscle strength, balance ability, and body flexibility. For example, the gait event index is the center of pressure excursion index (CPEI), which is an evaluation index of pronation/supination of the foot.

From the gait waveform, the feature amount of the gait phase cluster configured by the temporally continuous gait phase and the feature amount of the single gait phase are extracted. The feature amount extracted from the gait waveform is associated with the gait event index. That is, for the same subject or the same subject group, covariance can be used as an index indicating that the feature amount extracted from the gait waveform and the gait event index are relevant to each other. For example, a gait phase cluster GC0 in which temporally continuous gait phases i to i+4 are integrated is configured (i is a natural number). A feature amount is extracted from each of the gait phases i to i+4. The feature amount constitutive expression is applied to the feature amount extracted from each of the gait phases i to i+4, and the feature amount of the gait phase cluster GC0 is generated. The feature amount constitutive expression is a calculation expression set in advance in order to generate the feature amount of the gait phase cluster. For example, the feature amount constitutive expression is a calculation expression relating to four arithmetic operations. For example, the feature amount constitutive expression is a calculation expression relating to an integral average, an arithmetic average, an inclination, and a variation. The feature amount is also extracted from the single gait phase j (j is a natural number). The feature amount constitutive expression may be applied without distinguishing the gait phases i to i+4 and the single gait phase j constituting the gait phase cluster. For example, in a case where the feature amount constitutive expression is applied to the single gait phase j, the inclination and the variation cannot be calculated, so that the feature amount constitutive expression for calculating the integral average, the arithmetic average, or the like may be used. The plurality of feature amounts extracted from the sensor data for one gait cycle is integrated as a feature amount vector associated with the one gait cycle. For example, the plurality of feature amounts extracted from the gait waveform for one gait cycle is integrated using the feature amount constitutive expression.

The measurement data processing unit 112 acquires a plurality of pieces of pair data including the measurement gait data and the response variable relevant to the measurement gait data. The measurement data processing unit 112 extracts a feature amount from the measurement gait data. The measurement data processing unit 112 calculates a feature amount vector for each pair data based on the feature amount extracted from the measurement gait data. The feature amount vector is a vector obtained by combining feature amounts extracted for each piece of pair data. The feature amount vector is configured by a feature amount extracted for each gait data for one gait cycle.

The measurement data processing unit 112 generates a vector (also referred to as a measurement dataset vector) obtained by combining a feature amount vector and a response variable for each of the plurality of pieces of acquired pair data.

The measurement data processing unit 112 calculates an average vector of feature amount vectors for a plurality of pieces of pair data. For example, the measurement data processing unit 112 calculates an average value of feature amounts constituting a feature amount vector of the gait data for each gait phase with respect to a plurality of pieces of pair data. The measurement data processing unit 112 combines the average values of the feature amounts calculated for each gait phase to generate an average vector of the feature amount vectors. The measurement data processing unit 112 calculates an average value of the response variables for a plurality of pieces of pair data. The numerical value of the feature amount constituting the one-dimensional feature amount vector follows the Gaussian distribution. In the present example embodiment, since covariance is used as an index indicating that the feature amount extracted from the gait waveform is relevant to the gait event index (response variable), an addition average value is used as an average value of the feature amount and the response variable. When a dispersion other than the covariance is used, an average value relevant to the used dispersion may be used.

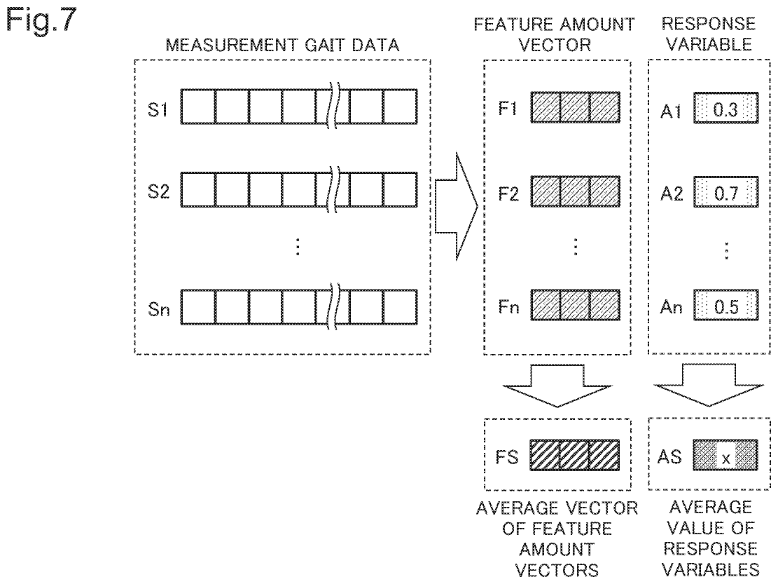

FIG. 7 is a conceptual diagram for explaining a feature amount vector F extracted from a plurality of pieces of measurement gait data S and a response variable A relevant to the feature amount vector F. From each of the plurality of pieces of measurement gait data S1 to Sn, each of the plurality of feature amount vectors F1 to Fn is extracted (n is a natural number). A vector obtained by combining the average values of the plurality of feature amounts constituting the plurality of feature amount vectors F1 to Fn is an average vector FS of the feature amount vectors F. Each of the plurality of feature amount vectors F1 to Fn is associated with each of the plurality of response variables A1 to An. FIG. 7 illustrates numerical values such as 0.3, 0.7, and 0.5 to indicate that the response variables A1 to An are numerical values. An average value of the plurality of response variables A1 to An is an average value AS of the response variables. In FIG. 7, a numerical value of the average value AS of the response variables is indicated by x (x is a real number).

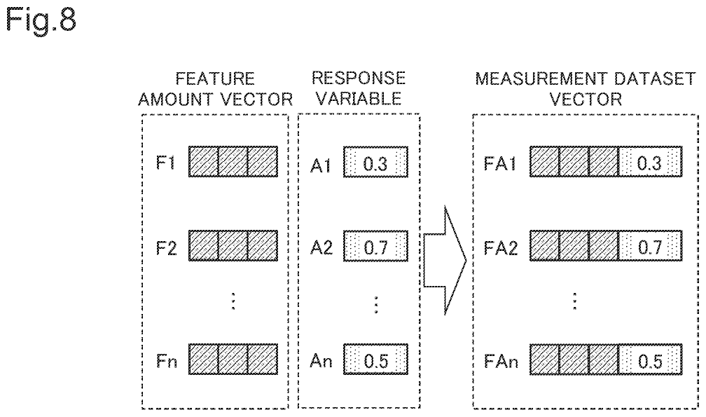

FIG. 8 is an example of generating a measurement dataset vector FA in which the feature amount vector F and the response variable A are combined. The measurement data processing unit 112 generates measurement dataset vectors FA1 to FAn in which a plurality of response variables A1 to An are associated with a plurality of feature amount vectors F1 to Fn, respectively.

The measurement data processing unit 112 generates a covariance matrix of a plurality of feature amounts and response variables for a plurality of measurement dataset vectors. When the measurement data processing unit 112 regards each of the plurality of measurement dataset vectors as a vector in one linear space, it can be considered that a vector group represented by all the measurement dataset vectors follows a multidimensional normal distribution in this linear space. Therefore, the measurement data processing unit 112 generates a covariance matrix relating to the measurement dataset vector. There is covariance between each of the plurality of feature amounts constituting the measurement dataset vector and the response variable. In some cases, there is covariance between a plurality of feature amounts constituting the dataset vector. A feature amount vector can be formed by a combination of some feature amounts. However, since all the feature amounts are associated with the response variable, the accuracy of covariance is higher when all the feature amounts are combined than when some feature amounts are combined. There is a limitation condition that the number of dimensions obtained by adding one to the number of feature amounts needs to be smaller than the number of pieces of data. Therefore, in a case where the number of pieces of data is small, the accuracy of covariance may be improved by removing feature amounts having low association or combining feature amounts having high association.

The measurement data processing unit 112 performs Cholesky decomposition on the covariance matrix to derive an upper triangular matrix of the covariance matrix. The measurement data processing unit 112 extracts a column vector at the end of the derived upper triangular matrix. Portions related to the response variable are collected in the column vector at the end of the upper triangular matrix.

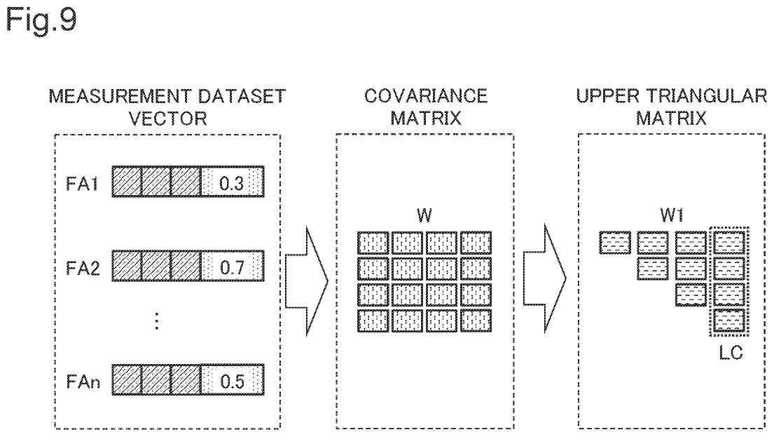

FIG. 9 is a conceptual diagram illustrating an example of deriving an upper triangular matrix W1 of a covariance matrix W for a plurality of measurement dataset vectors. The measurement data processing unit 112 calculates a covariance matrix W relating to a plurality of measurement dataset vectors FA1 to FAn. The measurement data processing unit 112 performs Cholesky decomposition on the calculated covariance matrix W to calculate the upper triangular matrix W1. A column vector LC at the end of the upper triangular matrix W1 is used for calculation of a deviation C of the pseudo response variable to be described later.

The pseudo data generation unit 113 acquires measurement gait data of a plurality of pieces of pair data from the acquisition unit 111. The pseudo data generation unit 113 acquires the average vector of the feature amount vectors and the average value of the response variables from the measurement data processing unit 112. The average value of the feature amount vectors and the average value of the response variables may be calculated by the pseudo data generation unit 113. A feature amount can be extracted from the measurement gait data and the pseudo gait data by a similar method.

The pseudo data generation unit 113 generates a gait waveform (also referred to as pseudo gait data) using the plurality of pieces of acquired measurement gait data. For example, the pseudo data generation unit 113 generates the pseudo gait data by adding temporal fluctuation or noise to the measurement gait data. For example, the pseudo data generation unit 113 can generate the pseudo gait data based on the measurement gait data using the method disclosed in NPL 1 (NPL 1: L. Tran and D. Choi, “Data Augmentation for Inertial Sensor-Based Gait Deep Neural Network”, IEEE Access, Vol. 8, pp. 12364-12378 (2020)).

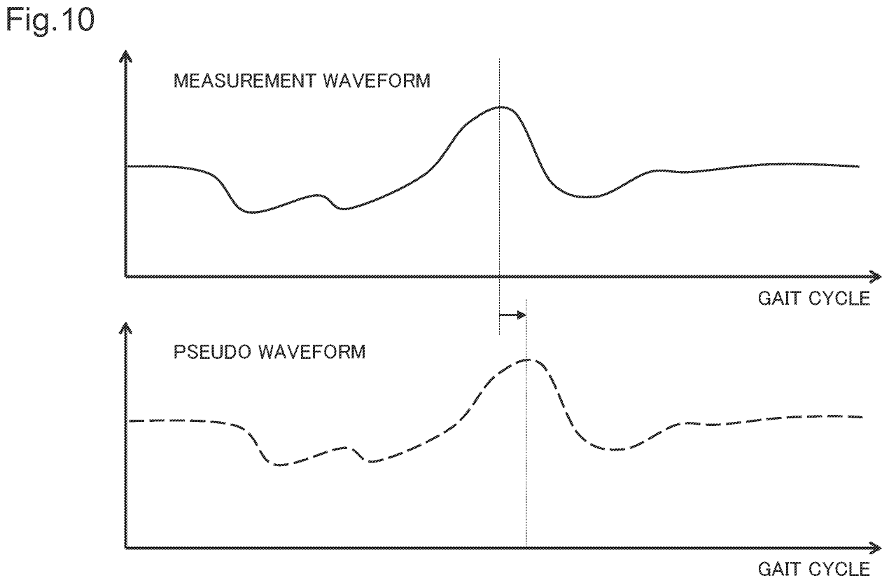

FIG. 10 is a conceptual diagram illustrating an example of generating time-series data (also referred to as a pseudo waveform) of the pseudo gait data based on time-series data (also referred to as a measured waveform) of the measurement gait data included in the pair data. The example of FIG. 10 is an example of generating a pseudo waveform by shifting at least a part of the measured waveform along the axis of the gait cycle and adding temporal fluctuation. For example, the pseudo waveform may be generated by shifting at least a part of the measured waveform in a direction perpendicular to or oblique to the axis of the gait cycle. For example, a noise level variation may be added to at least a part of the measured waveform to generate a pseudo waveform.

The pseudo data generation unit 113 extracts a feature amount (also referred to as a pseudo feature amount) from each of a plurality of pseudo waveforms (also referred to as pseudo gait data). The pseudo data generation unit 113 calculates a feature amount vector (also referred to as a pseudo feature amount vector) for each pseudo waveform based on the pseudo feature amount extracted from the pseudo waveform. The pseudo feature amount vector is a vector obtained by combining pseudo feature amounts extracted from the pseudo waveform. The pseudo feature amount vector is configured by a pseudo feature amount extracted for each pseudo waveform for one gait cycle.

In the case of the same subject or the same subject group, it is expected that the pseudo gait data also follows a distribution similar to that of the measurement gait data. That is, when the pseudo response variable relevant to the pseudo feature amount is calculated, the covariance between the pseudo feature amount and the pseudo response variable may be used. In the present example embodiment, it is assumed that the pseudo gait data has a covariance similar to that of the measurement gait data. In the present example embodiment, the pseudo response variable relevant to the pseudo feature amount is determined using the pseudo feature amount calculated from the pseudo gait data and the covariance between the feature amount calculated from the measurement gait data and the response variable.

The pseudo data generation unit 113 calculates a deviation vector (also referred to as a pseudo deviation vector) of the pseudo feature amount vector for each pseudo waveform by subtracting an average vector of the feature amount vectors from the pseudo feature amount vector for each of the plurality of pseudo waveforms.

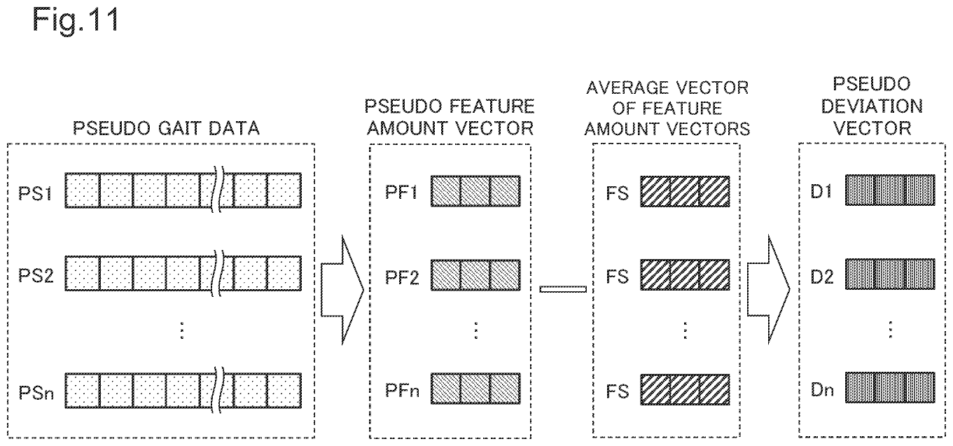

FIG. 11 is a conceptual diagram for explaining an example of generating a pseudo deviation vector D based on pseudo gait data PS. The pseudo data generation unit 113 extracts each of the plurality of pseudo feature amount vectors PF1 to PFn from each of the plurality of pieces of pseudo gait data PSI to PSn (n is a natural number). The pseudo data generation unit 113 calculates each of the pseudo deviation vectors D1 to Dn by subtracting the average vector FS of the plurality of feature amount vectors F1 to Fn from each of the plurality of pseudo feature amount vectors PF1 to PFn.

The pseudo data generation unit 113 generates a random numerical value according to a normal distribution of average value 0/standard deviation 1. The generated random numerical value is relevant to the dispersion generated by the random processing. The pseudo data generation unit 113 generates a pseudo variance vector for each of the plurality of pseudo waveforms by adding the generated random numerical value to the end of the pseudo deviation vector extracted for each of the plurality of pseudo waveforms.

The pseudo data generation unit 113 multiplies the generated pseudo variance vector for each of the plurality of pieces of pseudo gait data by the column vector at the end of the upper triangular matrix obtained from the covariance matrix, and calculates the deviation of the pseudo response variable relevant to each of the plurality of pieces of pseudo gait data.

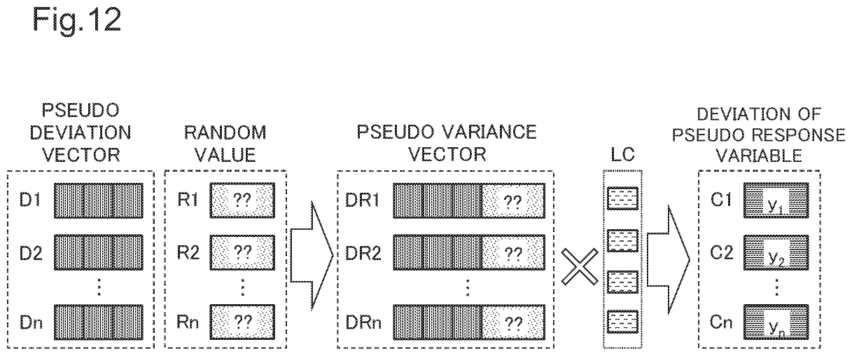

FIG. 12 is a conceptual diagram for explaining an example of calculating the deviation C of the pseudo response variable based on the pseudo deviation vector D extracted from the plurality of pieces of pseudo gait data PS. The pseudo data generation unit 113 generates random numerical values RI to Rn according to a normal distribution of average value 0/standard deviation 1 (n is a natural number). The pseudo data generation unit 113 adds each of the random numerical values RI to Rn to the end of each of the pseudo deviation vectors D1 to Dn of the pseudo feature amount vectors PF1 to PFn, thereby generating pseudo variance vectors DR1 to DRn for each of the plurality of pieces of pseudo gait data PS. The pseudo data generation unit 113 multiplies each of the plurality of generated pseudo variance vectors DR1 to DRn by the column vector LC at the end of the upper triangular matrix W1 obtained by Cholesky decomposition of the covariance matrix W to calculate the deviations Cl to Cn of the pseudo response variables. The numerical values of the deviations Cl to Cn of the pseudo response variable are y₁ to yₙ (y₁ to yₙ are real numbers).

The pseudo data generation unit 113 adds the average value of the response variables to the deviation of the pseudo response variable relevant to each of the plurality of pieces of pseudo gait data to calculate the pseudo response variable for each pseudo waveform. The pseudo data generation unit 113 generates a pseudo dataset vector in which a plurality of pseudo response variables are associated with the pseudo feature amount vector extracted for each of the plurality of pseudo waveforms.

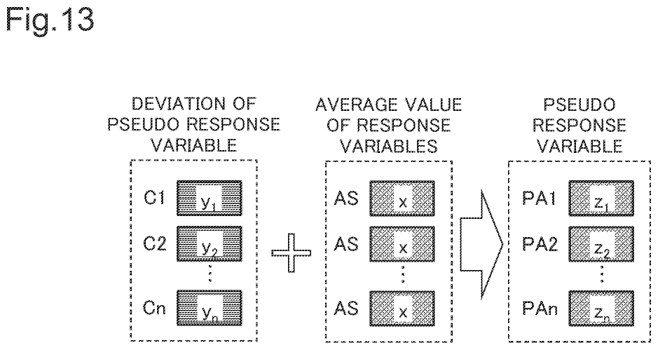

FIG. 13 is a conceptual diagram illustrating an example of calculating the pseudo response variable PA for each of the plurality of pieces of pseudo gait data PS using the deviation C of the pseudo response variable calculated for the plurality of pieces of pseudo gait data PS and the average value AS of the response variables calculated for the plurality of pieces of pair data. The pseudo data generation unit 113 adds the average value AS of the response variables to the deviations Cl to Cn of the pseudo response variables relevant to each of the plurality of pieces of pseudo gait data PSI to PSn to generate pseudo response variables PA1 to PAn (n is a natural number).

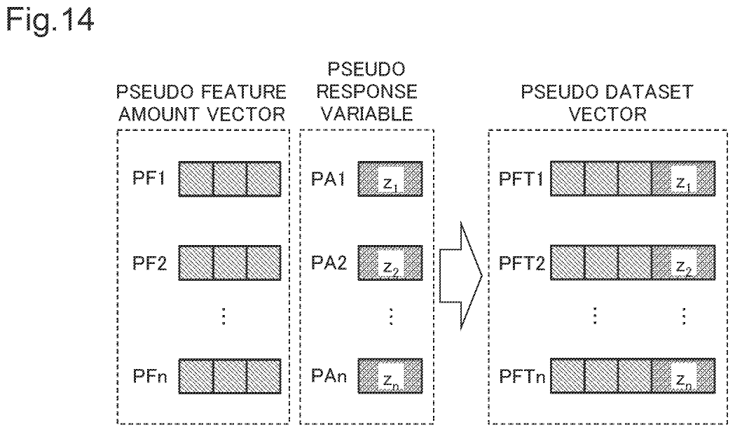

FIG. 14 is a conceptual diagram illustrating an example of generating a pseudo dataset vector PFT by associating a plurality of pseudo response variables PA with a pseudo feature amount vector PF extracted for each of a plurality of pieces of pseudo gait data PS. The pseudo data generation unit 113 generates pseudo dataset vectors PTF1 to PTFn in which each of the plurality of pseudo response variables PA1 to PAn is associated with the pseudo feature amount vectors PF1 to PFn extracted for each of the plurality of pieces of pseudo gait data PSI to PSn (n is a natural number).

The output unit 115 outputs a plurality of datasets configured by the measurement dataset vector generated by the measurement data processing unit 112 and the pseudo dataset vector generated by the pseudo data generation unit 113 to the learning device 15. The plurality of datasets output from the output unit 115 is used for generation of an estimation model by the learning device 15.

As described above, the data generation device 11 of the present example embodiment can extend the dataset used for generation of the estimation model using the pair data including the gait data based on the actually measured sensor data. With the use of the method of NPL 1, when the response variable is fixed, the dataset can be extended. However, in the method of NPL 1, when the response variable is a variable, the dataset cannot be appropriately extended. Even in a case where the response variable is a variable, the data generation device 11 of the present example embodiment can calculate the pseudo response variable according to the pseudo feature amount vector extracted from the pseudo gait data, so that the dataset can be appropriately extended.

In the configuration of FIG. 1, the measurement dataset vector is generated by the measurement data processing unit 112, and the pseudo dataset vector is generated by the pseudo data generation unit 113. For example, a common feature amount extraction unit (not illustrated) may be configured to extract a feature amount (pseudo feature amount) from a pseudo waveform generated based on a measured waveform and a measured waveform. In that case, the pseudo response variable may be generated based on the pseudo feature amount using the above-described method. For example, a dataset vector may be generated in a common dataset vector generation unit (not illustrated) for the pseudo gait data and the measurement gait data.

The expansion of the dataset by the data generation device 11 can be performed at an arbitrary timing. For example, the data generation device 11 can extend the dataset using the pair data accumulated in the past. For example, the data generation device 11 can extend the dataset in real time in accordance with the measurement of the sensor data (gait data) in addition to the pair data accumulated in the past.

### [Learning Device]

The learning device 15 of the learning system 10 will be described. FIG. 15 is a block diagram illustrating an example of a configuration of the learning device 15. The learning device 15 includes a dataset acquisition unit 151, a learning unit 153, and a storage unit 155.

The dataset acquisition unit 151 acquires a plurality of datasets generated by the data generation device 11. The plurality of datasets include measurement dataset vectors and pseudo dataset vectors. The dataset acquisition unit 151 stores the acquired dataset in the storage unit 155. The measurement dataset vector and the pseudo dataset vector may be stored in the storage unit 155 so as to be distinguishable, or may be stored in the storage unit 155 without distinction.

The learning unit 153 acquires a plurality of datasets from the storage unit 155. The learning unit 153 extracts a feature amount vector and a response variable from a plurality of datasets. When the dataset vector is a measurement dataset vector, the learning unit 153 extracts a feature amount vector and a response variable. When the dataset vector is a pseudo dataset vector, the learning unit 153 extracts a pseudo feature amount vector and a pseudo response variable. Hereinafter, the pseudo feature amount vector and the pseudo response variable extracted from the pseudo dataset vector will be referred to as a feature amount vector and a response variable without being distinguished from the feature amount vector and the response variable extracted from the measurement dataset vector.

The learning unit 153 generates an estimation model using the feature amount vectors and the response variables of the plurality of datasets. The learning unit 153 generates an estimation model that outputs a response variable (gait event index) in response to the input of the gait data. For example, the learning unit 153 uses some of the plurality of datasets as learning data (training data) and uses the remaining some as verification data or test data.

For example, the learning unit 153 may generate an estimation model dedicated to the user using a feature amount vector and a response variable of a dataset generated based on pair data measured in accordance with walking of the user. For example, a personal identifier for uniquely specifying the user who is the acquisition source of the sensor data is given to the sensor data, and the estimation model may be generated using the sensor data to which the same personal identifier is given. Even if the estimation model dedicated to the user does not have versatility for all people, the physical condition can be accurately estimated by being specialized for the user.

For example, the learning unit 153 may generate the estimation model using the feature amount vector and the response variable of the dataset generated based on the pair data measured in accordance with attributes such as gender, age, weight, and height. Even if the estimation model according to the attribute does not have versatility for all the attributes, the physical condition can be accurately estimated by being specialized for the user of the attribute.

For example, the learning unit 153 may generate the estimation model using the feature amount vector and the response variable of the dataset generated based on the pair data measured in accordance with the characteristic symptom such as the pain of the knee or the foot. Even if the estimation model according to the symptom does not have versatility for all the symptoms, the physical condition can be accurately estimated by focusing on the symptom.

The storage unit 155 stores a plurality of datasets. The storage unit 155 stores the estimation model generated by the learning unit 153. The estimation model stored in the storage unit 155 is implemented in an estimation device (not illustrated) that estimates the physical condition of the user using the gait data of the user. A method for implementing the estimation model in the estimation device is not particularly limited.

The learning using the dataset by the learning device 15 can be performed at an arbitrary timing. For example, the learning device 15 can execute learning using a dataset extended using pair data accumulated in the past. For example, the data generation device 11 can execute learning using a dataset extended in real time in accordance with measurement of sensor data (gait data) in addition to the pair data accumulated in the past. When learning is executed using the dataset extended in real time, an estimation model in which the user's current physical condition is more reflected can be constructed.

### (Operation)

Next, an operation of the learning system 10 will be described with reference to the drawings. Hereinafter, the operations of the data generation device 11 and the learning device 15 included in the learning system 10 will be individually described.

### [Data Generation Device]

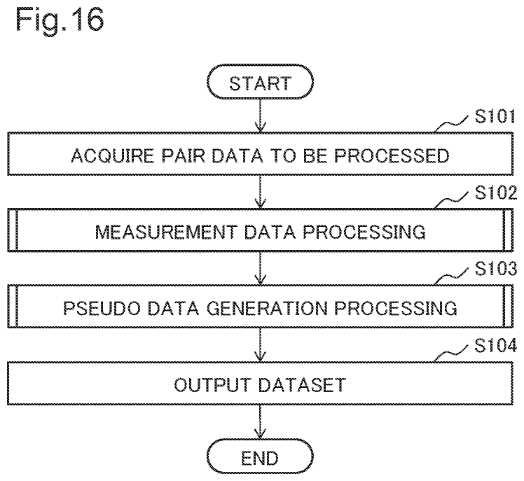

First, the operation of the data generation device 11 will be described with reference to the drawings. FIG. 16 is a flowchart for explaining an example of the operation of the data generation device 11. In the description along the flowchart of FIG. 16, the data generation device 11 will be described as an operation subject.

In FIG. 16, first, the data generation device 11 acquires pair data to be processed (step S101). For example, the data generation device 11 acquires pair data accumulated in a cloud or a server. For example, the data generation device 11 may be configured to directly acquire the pair data from the measurement device 110 installed on the foot of the user.

Next, the data generation device 11 executes measurement data processing on the pair data to be processed (step S102). In the measurement data processing, the data generation device 11 generates a measurement dataset vector based on a dataset to be processed. Details of the measurement data processing will be described later.

Next, the data generation device 11 executes pseudo data generation processing on the dataset to be processed (step S103). In the pseudo data generation processing, the data generation device 11 generates a pseudo dataset vector based on a dataset to be processed. Details of the pseudo data generation processing will be described later.

Next, the data generation device 11 outputs the generated dataset (measurement dataset vector/pseudo dataset vector) (step S104). The output dataset is used for learning by the learning device 15.

### [Measurement Data Processing]

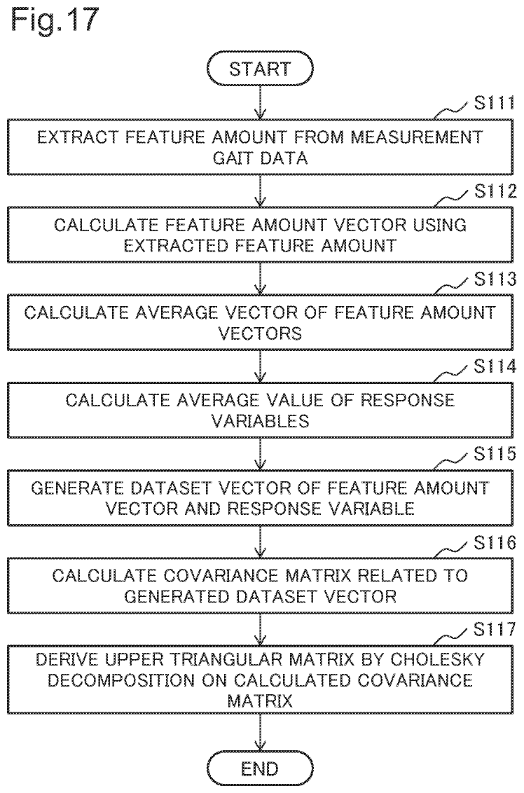

Next, the measurement data processing (step S102 in FIG. 16) by the data generation device 11 will be described with reference to the drawings. FIG. 17 is a flowchart for explaining an example of measurement data processing by the data generation device 11. In the description along the flowchart of FIG. 17, the data generation device 11 will be described as an operation subject. Vectors and numerical values derived in the description along the flowchart of FIG. 17 are stored in a storage unit (not illustrated).

In FIG. 17, first, the data generation device 11 extracts a feature amount from the measurement gait data included in the pair data to be processed (step S111).

Next, the data generation device 11 generates a feature amount vector for each pair data using the extracted feature amount (step S112).

Next, the data generation device 11 calculates an average vector of feature amount vectors generated with respect to the measurement gait data for each piece of pair data to be processed (step S113). The calculated average vector is used in pseudo data generation processing to be described later.

Next, the data generation device 11 calculates an average value of the response variables relating to the plurality of pieces of measurement gait data for the dataset to be processed (step S114). The calculated average value of the response variables is used in pseudo data generation processing to be described later.

Next, the data generation device 11 generates a feature amount vector for each piece of pair data and a measurement dataset vector of a response variable (step S115). The generated measurement dataset vector is output as a dataset in step S104 of FIG. 16.

Next, the data generation device 11 generates a covariance matrix relating to the generated measurement dataset vector (step S116).

Next, the data generation device 11 performs Cholesky decomposition on the calculated covariance matrix to derive an upper triangular matrix (step S117). A column at the end of the upper triangular matrix derived by Cholesky decomposition of the covariance matrix is used in pseudo data generation processing to be described later.

### [Pseudo Data Generation Processing]

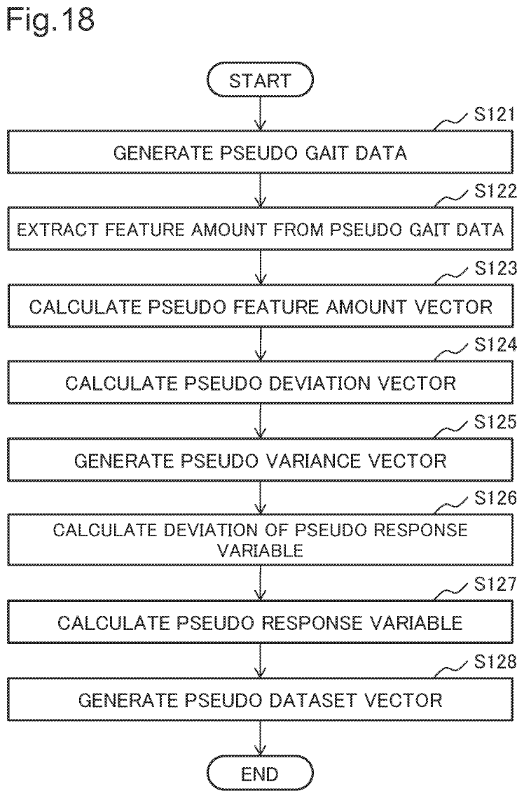

Next, the pseudo data generation processing (step S103 in FIG. 16) by the data generation device 11 will be described with reference to the drawings. FIG. 18 is a flowchart for explaining an example of pseudo data generation processing by the data generation device 11. In the description along the flowchart of FIG. 18, the data generation device 11 will be described as an operation subject. Vectors and numerical values derived in the description along the flowchart of FIG. 18 are stored in a storage unit (not illustrated).

In FIG. 18, first, the data generation device 11 generates the pseudo gait data using the measurement gait data included in the pair data to be processed (step S121).

Next, the data generation device 11 extracts a pseudo feature amount from the generated pseudo gait data (step S122).

Next, the data generation device 11 calculates a pseudo feature amount vector for each piece of pseudo gait data using the extracted pseudo feature amount (step S123).

Next, the data generation device 11 calculates a pseudo deviation vector using the pseudo feature amount vector and the average feature amount vector for each piece of pseudo gait data (step S124). The data generation device 11 calculates a pseudo deviation vector by subtracting the average feature amount vector from the pseudo feature amount vector.

Next, the data generation device 11 adds a random value to the end of the pseudo deviation vector for each piece of pseudo gait data to generate a pseudo variance vector (step S125).

Next, the data generation device 11 multiplies the pseudo variance vector for each piece of pseudo gait data by the column at the end of the upper triangular matrix derived by Cholesky decomposition of the covariance matrix to calculate a deviation of the pseudo response variable (step S126).

Next, the data generation device 11 calculates the pseudo response variable by adding the average value of the response variables to the deviation of the pseudo response variable for each piece of pseudo gait data (step S127).

Next, the data generation device 11 generates a pseudo dataset vector by combining the pseudo feature amount for each piece of pseudo gait data and the pseudo response variable (step S128). The generated pseudo dataset vector is output as a dataset in step S104 of FIG. 16.

### [Learning Device]

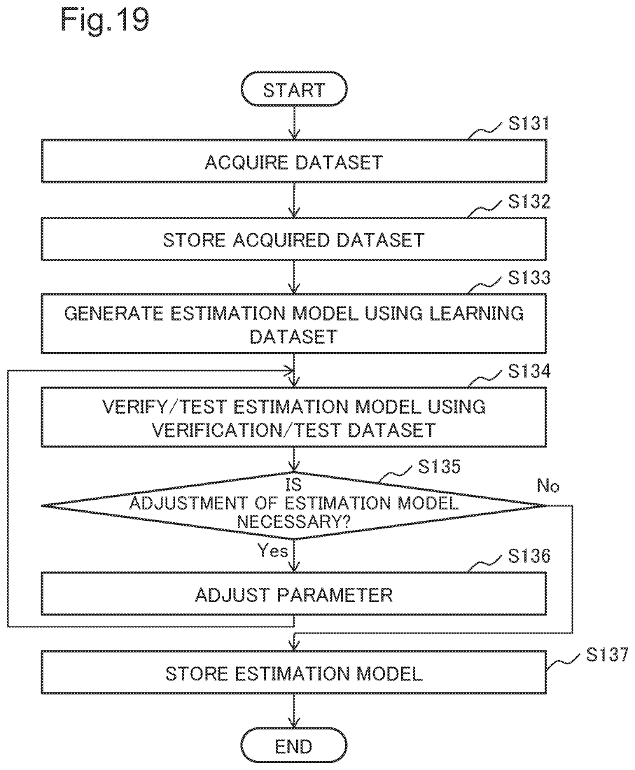

Next, the operation of the learning device 15 will be described with reference to the drawings. FIG. 19 is a flowchart for explaining an example of the operation of the learning device 15. In the description along the flowchart of FIG. 19, the learning device 15 will be described as an operation subject.

In FIG. 16, first, the learning device 15 acquires a dataset generated by the data generation device 11 (step S131). The dataset includes a measurement dataset vector and a pseudo dataset vector.

Next, the learning device 15 stores the acquired dataset (step S132). The datasets are classified for learning, validation, and testing.

Next, the learning device 15 generates an estimation model using a learning dataset among the stored datasets (step S133).

Next, the learning device 15 verifies/tests the estimation model using a verification/test dataset among the stored datasets (step S134). For example, the learning device 15 executes a test using the test dataset following the verification using the verification dataset. For example, the learning device 15 performs cross verification such as k-division, hold-out, leave-one-out, random iterative subsampling, layering, and re-substitution using the verification dataset.

In a case where adjustment of the estimation model is necessary (Yes in step S135), the learning device 15 adjusts parameters of the estimation model (step S136). For example, the learning device 15 adjusts the parameters of the estimation model in a case where a predetermined index such as accuracy, a generalization error, an accuracy rate, a matching rate, a reproduction rate, or an F value is less than the reference. After step S136, the process proceeds to step S134.

When the adjustment of the estimation model is not necessary (No in step S135), the learning device 15 stores the estimation model (step S137). For example, in a case where a predetermined index such as accuracy, a generalization error, an accuracy rate, a matching rate, a reproduction rate, or an F value satisfies the reference, the learning device 15 stores the estimation model. The estimation model generated by the learning device 15 is implemented in an estimation device (not illustrated) that estimates a physical condition using the gait data.

### [Evaluation Example of Estimation Model]

Next, a result of evaluating the estimation model generated using the method of the present example embodiment will be described with an example. Hereinafter, a result of evaluating the correlation between the true value of the response variable included in the dataset and the estimated value of the response variable obtained by inputting the measurement gait data included in the dataset to the estimation device using the dataset including the actually measured gait data will be described.

First, improvement of the intraclass correlation coefficients ICC by data extension will be described. Here, CPEI (Center of Pressure Excursion Index), which is an evaluation index of pronation/supination of the foot, has been used as the response variable. The original measurement dataset vector is 500 steps for 32 men. In this evaluation, by adding a random temporal fluctuation to the measurement gait data for each step included in the original measurement dataset vector, pseudo gait data for 6000 steps has been generated for 32 men. By LOSO (Leave-one-subject-out), a measurement dataset vector of one person has been used for a test, and a measurement dataset vector of another person and a pseudo dataset vector have been combined to generate an estimation model. LOSO has been performed on all 32 subjects and all test predictive and true values have been compared together.

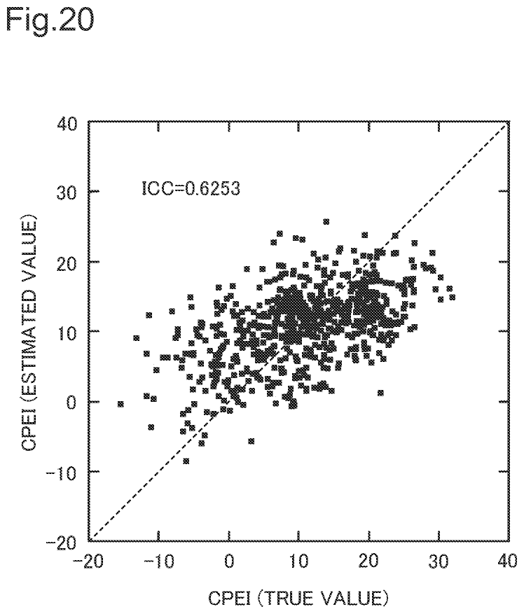

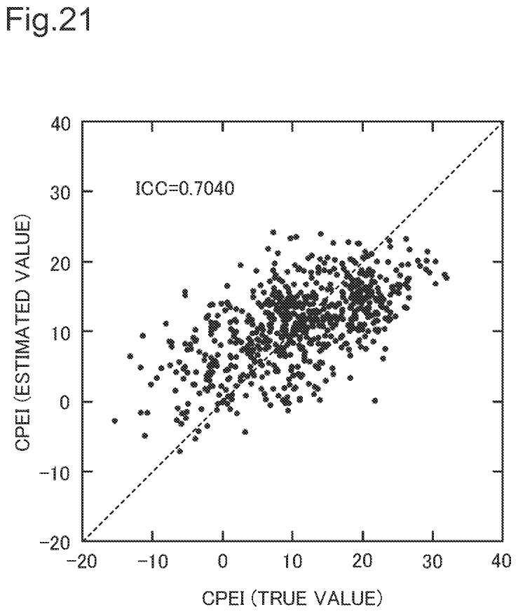

FIG. 20 is a graph illustrating a correlation between a true value of CPEI and an estimated value in a case where data extension is not performed. In the example of FIG. 20, the measurement dataset vector is used as it is. In the example of FIG. 20, the intraclass correlation coefficient ICC between the true value of CPEI and the estimated value has been 0.6253. FIG. 21 is a graph illustrating a correlation between a true value of CPEI and an estimated value in a case where data extension is performed. In the example of FIG. 21, a measurement dataset vector and a pseudo measurement dataset vector are used. In the example of FIG. 21, the intraclass correlation coefficient ICC between the true value of CPEI and the estimated value has been 0.7040. In the examples of FIGS. 20 and 21, the true value of CPEI and the value of the intraclass correlation coefficient ICC of the estimated value are better in the data extension. That is, it has been possible to construct a highly accurate estimation model by data expansion.

Next, results of evaluating an estimation model generated using the measurement dataset vectors of 32 men on the measurement dataset vectors of 12 men different from those men are shown.

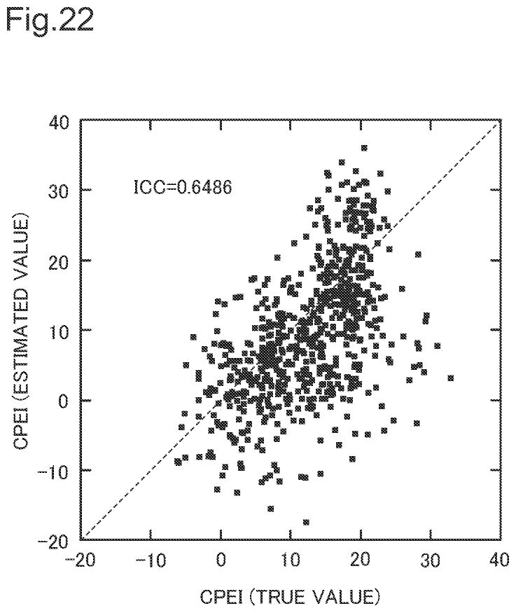

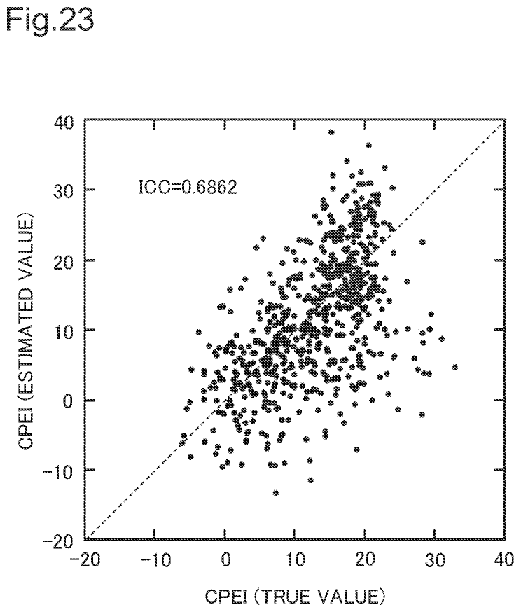

FIG. 22 is a graph illustrating a correlation between a true value of CPEI and an estimated value in a case where an estimation model without data extension (estimation model in FIG. 20) is used. In the example of FIG. 22, the intraclass correlation coefficient ICC between the true value of CPEI and the estimated value has been 0.6486. FIG. 23 is a graph illustrating a correlation between a true value of CPEI and an estimated value in a case where a data-extended estimation model (estimation model in FIG. 21) is used. In the example of FIG. 23, the intraclass correlation coefficient ICC between the true value of CPEI and the estimated value has been 0.6862. In the examples of FIGS. 22 and 23, the intraclass correlation coefficient ICC between the true value of CPEI and the estimated value are better when the data-extended estimation model is used. That is, the estimation model having high versatility can be constructed by the data extension.

### APPLICATION EXAMPLE

Next, an application example of the present example embodiment will be described with reference to the drawings. In the application example of the present example embodiment, the measurement device is installed in the shoe of the user, and the gait data measured by the measurement device is transmitted to the mobile terminal possessed by the user. The sensor data transmitted to the mobile terminal is processed by an estimation device implemented in the mobile terminal. For example, the function of the estimation device is provided as an application that can be installed in a mobile terminal.

### Application Example 1-1

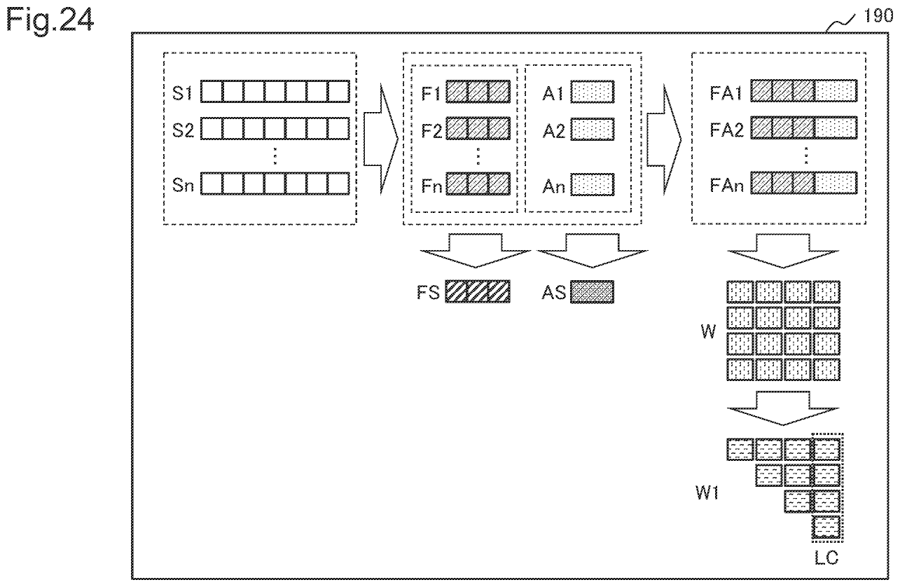

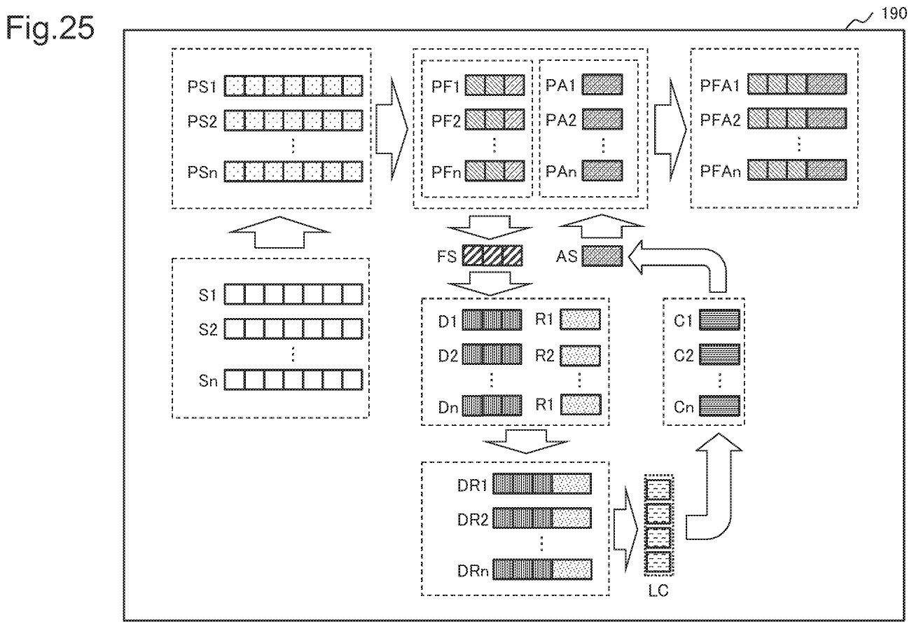

FIGS. 24 and 25 are conceptual diagrams for explaining Application Example 1-1. In the present application example, an algorithm for generating a dataset using the measurement gait data is visualized and displayed on a screen of a terminal device visible by the user.

FIG. 24 is an example in which an algorithm for generating the measurement dataset vectors FA1 to FAn using the measurement gait data S1 to Sn is visualized and displayed on a screen 190 of the terminal device visible by the user. In the example of FIG. 24, the average vector FS of the feature amount vectors F1 to Fn extracted from the measurement gait data S1 to Sn and the average value AS of the response variables A1 to An are also displayed on the screen 190. In the example of FIG. 24, the covariance matrix W relating to the measurement dataset vectors FA1 to FAn and the upper triangular matrix W1 derived by Cholesky decomposition of the covariance matrix W are also displayed on the screen 190. By referring to the algorithm visualized on the screen 190, the process of generating the measurement dataset vectors FA1 to FAn, the covariance matrix, and the like can be intuitively grasped using the measurement gait data S1 to Sn.

FIG. 25 is an example in which an algorithm for generating pseudo dataset vectors PFA1 to PFAn using the measurement gait data S1 to Sn is visualized and displayed on the screen 190 of a terminal device visible by the user. In FIG. 25, a process of calculating the pseudo feature amount vectors PF1 to PFn, the pseudo deviation vectors D1 to Dn, the pseudo variance vectors DR1 to DRn, and the deviations Cl to Cn of the pseudo response variables and calculating the pseudo response variables PA1 to PAn is displayed on the screen 190. In FIG. 25, the average vector FS of the feature amount vectors F1 to Fn, the average value AS of the response variables A1 to An, and the column vector LC at the end of the upper triangular matrix W1 used to calculate the pseudo response variables PA1 to PAn are also displayed on the screen 190. By referring to the algorithm visualized on the screen 190, it is possible to intuitively grasp the process of generating the pseudo dataset vectors PFA1 to PFAn using the measurement gait data S1 to Sn. For example, in a case where there is a problem in the accuracy or versatility of the estimation model generated by learning using the dataset, it is possible to visually verify which process has a problem by referring to the algorithm displayed on the screen 190.

### Application Example 1-2

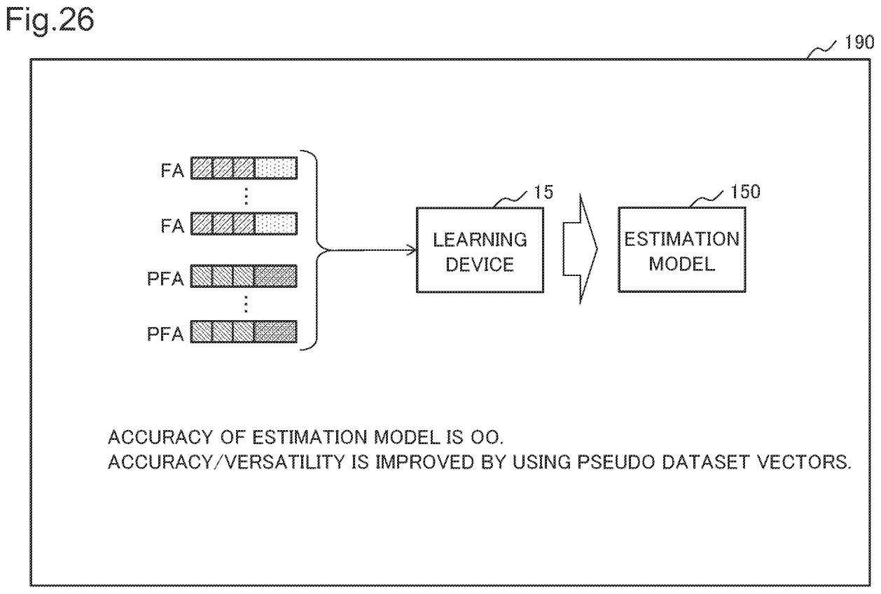

FIG. 26 is a conceptual diagram for explaining Application Example 1-2. In the present application example, information relating to learning using the dataset generated by the data generation device 11 is displayed on a screen of a terminal device visible by the user.

FIG. 26 is an example in which information relating to the accuracy and versatility of an estimation model 150 generated when the learning device 15 is caused to learn the dataset generated by the data generation device 11 is displayed on the screen of the terminal device visible by the user. In the example of FIG. 26, a state in which the measurement dataset vector FA and the pseudo dataset vector PSA generated by the data generation device 11 are learned by the learning device 15 and the estimation model 150 is generated is displayed on the screen 190. On the screen 190, information relating to the accuracy of “The accuracy of the estimation model is ◯◯.” is displayed as the accuracy of the estimation model. Information relating to versatility, such as “Accuracy/versatility is improved using pseudo dataset vectors.”, is also displayed on the screen 190.

According to the present application example, the effect of learning using the dataset generated by the data generation device 11 can be grasped in accordance with the information relating to the accuracy and versatility displayed on the screen 190.

As described above, the learning system of the present example embodiment includes the data generation device and the learning device.

The data generation device includes an acquisition unit, a measurement data processing unit, a pseudo data generation unit, and an output unit. The acquisition unit acquires pair data in which measurement gait data relating to sensor data measured in accordance with the movement of a user's feet and a response variable relevant to the measurement gait data are combined. The measurement data processing unit generates a measurement dataset vector by combining a feature amount vector calculated using a feature amount extracted from the measurement gait data and a response variable. The measurement data processing unit generates a covariance matrix relating to a plurality of pieces of pair data. The pseudo data generation unit generates pseudo gait data using the measurement gait data. The pseudo data generation unit generates a pseudo dataset vector by combining a pseudo feature amount vector calculated using a pseudo feature amount extracted from the pseudo gait data and a pseudo response variable generated using a covariance matrix relating to the pseudo feature amount vector. The output unit outputs a dataset including the measurement dataset vector and the pseudo dataset vector. The learning device acquires the dataset output from the data generation device. The learning device generates an estimation model that outputs a response variable according to the physical condition of the user in response to the input of the measurement gait data using the acquired dataset.

The gait data based on sensor data relating to foot movement includes specific features to gait events associated with health conditions. By generating a model (estimation model) in which learning data in which the gait data and the gait event relevant to the gait data are associated with each other is learned, the gait event can be estimated in accordance with the measured gait data. In order to generate the estimation model, it is necessary to collect learning data for learning the relevance between the gait data and the gait event. In order to generate an estimation model with sufficient accuracy, a large amount of learning data is required. It takes a lot of time and effort to collect a large amount of learning data. According to the method of NPL 1, a label such as an identifier can be given to the pseudo gait data. However, in the method of NPL 1, a numerical value (gait event index) according to the gait data cannot be associated with the pseudo gait data. According to the method of the present example embodiment, a numerical value (gait event index) according to the gait data can be generated in association with the pseudo gait data. That is, according to the method of the present example embodiment, it is possible to generate a dataset used for learning even when the response variable is a continuous value. Therefore, according to the method of the present example embodiment, it is possible to save time and effort spent on collection of learning data and construct a highly accurate estimation model.

In an aspect of the present example embodiment, the pseudo data generation unit generates a plurality of pieces of pseudo gait data by adding fluctuation to a plurality of pieces of measurement gait data included in a plurality of pieces of pair data. According to the present aspect, it is possible to increase the pseudo gait data by adding fluctuation to the plurality of pieces of measurement gait data.

In an aspect of the present example embodiment, the pseudo data generation unit generates a plurality of pieces of pseudo gait data by adding noise to a plurality of pieces of measurement gait data included in a plurality of pieces of pair data. According to the present aspect, it is possible to increase the pseudo gait data by adding noise to the plurality of pieces of measurement gait data.

In an aspect of the present example embodiment, the pseudo data generation unit generates a plurality of pieces of pseudo gait data using the measurement gait data. The pseudo data generation unit extracts at least one pseudo feature amount from the generated pseudo gait data. The pseudo data generation unit generates a pseudo feature amount vector for each piece of pseudo gait data using the pseudo feature amount extracted from the pseudo gait data. According to the present aspect, the feature amount vector used to generate the pseudo dataset vector can be generated for each piece of pseudo gait data.

In an aspect of the present example embodiment, the measurement data processing unit extracts at least one feature amount according to the gait event from the measurement gait data derived using the sensor data for each gait cycle. A feature amount vector is generated for each piece of measurement gait data using a feature amount extracted from the measurement gait data for each gait cycle. A response variable associated with the measurement gait data is added to the end of the feature amount vector generated for each piece of measurement gait data, and a measurement dataset vector for each piece of measurement gait data is generated. According to the present aspect, the measurement dataset vector can be generated for each data pair.

In an aspect of the present example embodiment, the measurement data processing unit calculates an average vector of a plurality of feature amount vectors calculated from a plurality of pieces of measurement gait data for a plurality of pieces of pair data. The measurement data processing unit generates a covariance matrix for a plurality of measurement dataset vectors generated for each piece of measurement gait data. The measurement data processing unit derives an upper triangular matrix of the covariance matrix by performing Cholesky decomposition on the generated covariance matrix. The measurement data processing unit calculates an average value of a plurality of response variables for a plurality of pieces of pair data. The pseudo data generation unit calculates a pseudo deviation vector of each of the plurality of pieces of pseudo gait data by subtracting an average vector of feature amounts from each of the plurality of pieces of pseudo gait data. The pseudo data generation unit generates a pseudo variance vector by adding a random value from 0 to 1 to the end of each of the plurality of calculated pseudo deviation vectors. The pseudo data generation unit integrates the column at the end of the upper triangular matrix with each of the plurality of generated pseudo variance vectors, and calculates a deviation of the pseudo response variable for each piece of pseudo gait data. The pseudo data generation unit calculates the pseudo response variable relevant to the pseudo gait data by adding the deviation of the pseudo response variable calculated for each piece of pseudo gait data to the average value of the response variables. According to the present aspect, the pseudo feature amount vector calculated using the pseudo feature amount extracted from the pseudo gait data and the pseudo response variable generated using the covariance matrix relating to the plurality of pieces of pair data can be combined to generate the pseudo dataset vector.

In an aspect of the present example embodiment, the output unit displays information relating to the generated dataset on the screen of the terminal device. According to the present aspect, the dataset generated by the data generation unit can be confirmed on the screen of the terminal device.

### Second Example Embodiment

Next, an estimation system according to a second example embodiment will be described with reference to the drawings. The estimation system of the present example embodiment estimates the physical condition of the user using the estimation model generated by the learning device according to the first example embodiment.

### (Configuration)

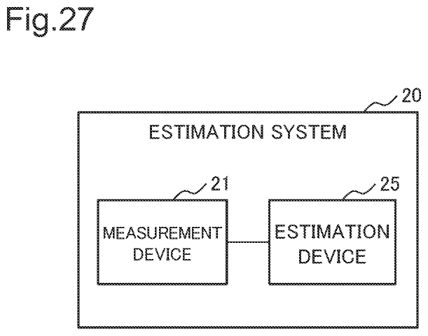

FIG. 27 is a block diagram illustrating a configuration of an estimation system 20 according to the present example embodiment. The estimation system 20 includes a measurement device 21 and an estimation device 25. For example, the estimation system 20 may be configured only by the estimation device 25 except for the measurement device 21. Hereinafter, the measurement device 21 and the estimation device 25 will be individually described.

### [Measurement Device]

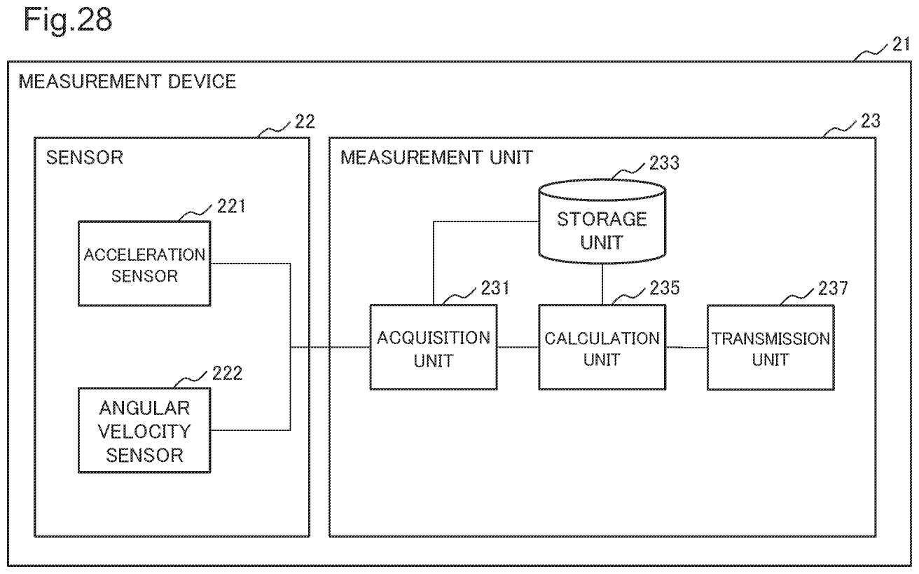

FIG. 28 is a block diagram illustrating an example of a configuration of the measurement device 21. The measurement device 21 includes a sensor 22 and a measurement unit 23. The sensor 22 includes an acceleration sensor 221 and an angular velocity sensor 222. The measurement unit 23 includes an acquisition unit 231, a storage unit 233, a calculation unit 235, and a transmission unit 237. The measurement device 21 is installed on the foot portion. For example, the measurement device 21 is similar to the first example embodiment in terms of a coordinate system and the like set in the measurement device 21 installed in footwear such as shoes.

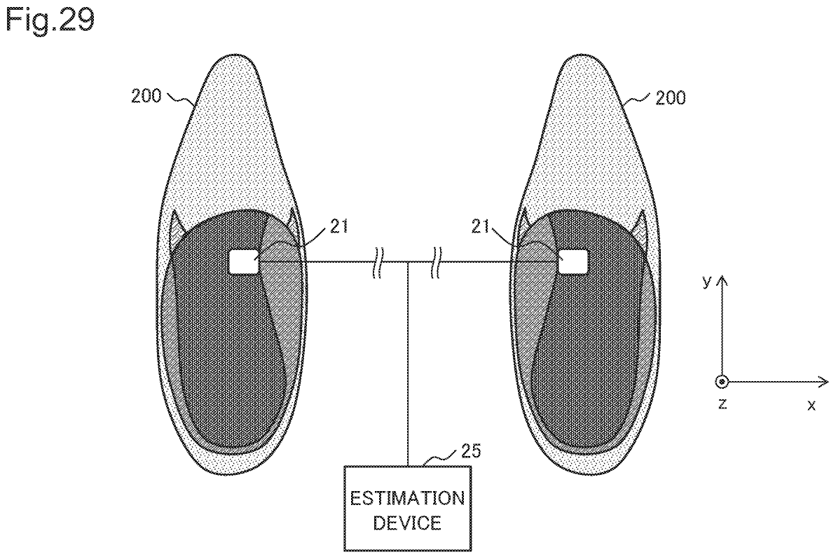

FIG. 29 is a conceptual diagram illustrating an example of the measurement device 21 disposed in a shoe 200. In the example of FIG. 29, the measurement device 21 is installed at a position relevant to the back side of the arch of foot. For example, the measurement device 21 is disposed in an insole inserted into the shoe 200. For example, the measurement device 21 is disposed on the bottom surface of the shoe 200. For example, the measurement device 21 is embedded in the main body of the shoe 200. The measurement device 21 may be detachable from the shoe 200 or may not be detachable from the shoe 200. The measurement device 21 may be disposed at a position other than the back side of the arch of foot as long as the sensor data relating to the movement of the feet can be acquired. The measurement device 21 may be installed on a sock worn by the user or a decorative article such as an anklet worn by the user. The measurement device 21 may be directly attached to the foot or may be embedded in the foot. FIG. 29 illustrates an example in which the measurement devices 21 are installed on the shoes 200 of both feet. The measurement device 21 may be installed in the shoe 200 of one foot.

The acceleration sensor 221 is a sensor that measures accelerations (also referred to as spatial accelerations) in three axial directions. The acceleration sensor 221 measures acceleration (also referred to as spatial acceleration) as a physical amount relating to the movement of the feet. The acceleration sensor 221 outputs the measured acceleration to the measurement unit 23. For example, a sensor of a piezoelectric type, a piezoresistive type, a capacitance type, or the like can be used as the acceleration sensor 221. The sensor used as the acceleration sensor 221 is not limited to the measurement type as long as the sensor can measure acceleration.

The angular velocity sensor 222 is a sensor that measures an angular velocity (also referred to as a spatial angular velocity) in the three axial directions. The angular velocity sensor 222 measures an angular velocity (also referred to as a spatial angular velocity) as a physical amount relating to the movement of the feet. The angular velocity sensor 222 outputs the measured acceleration to the measurement unit 23. For example, a sensor of a vibration type, a capacitance type, or the like can be used as the angular velocity sensor 222. The sensor used as the angular velocity sensor 222 is not limited to the measurement type as long as the sensor can measure the angular velocity.

The sensor 22 is achieved by, for example, an inertial measurement device that measures acceleration and angular velocity. An example of the inertial measurement device is an inertial measurement unit (IMU). The IMU includes an acceleration sensor 221 that measures accelerations in three axial directions and an angular velocity sensor 222 that measures angular velocities around three axes. The sensor 22 may be achieved by an inertial measurement device such as a vertical gyro (VG) or an attitude heading (AHRS). The sensor 22 may be achieved by Global Positioning System/Inertial Navigation System (GPS/INS). The sensor 22 may be achieved by a device other than the inertial measurement device as long as it can measure a physical amount relating to the movement of the feet.

The acquisition unit 231 acquires accelerations in three axial directions from the acceleration sensor 221. The acquisition unit 231 acquires angular velocities around three axes from the angular velocity sensor 222. The acquisition unit 231 converts the acquired acceleration and angular velocity into digital data, and stores the converted digital data (also referred to as sensor data) in the storage unit 233. The acquisition unit 231 may be configured to directly output the sensor data to the calculation unit 235. The sensor data includes at least acceleration data converted into digital data and angular velocity data converted into digital data. The acceleration data includes acceleration vectors in three axial directions. The angular velocity data includes angular velocity vectors around three axes. The acceleration data and the angular velocity data are associated with acquisition times of the data. The acquisition unit 231 may add correction such as a mounting error, temperature correction, and linearity correction to the acceleration data and the angular velocity data.

The storage unit 233 stores sensor data. The sensor data stored in the storage unit 233 is used for the calculation of the gait parameter by the calculation unit 235. In a case where the calculation of the gait parameter is omitted and the sensor data is directly transmitted to the estimation device 25, the sensor data stored in the storage unit 233 may be transmitted as it is from the transmission unit 237.

The calculation unit 235 acquires sensor data from the storage unit 233. The calculation unit 235 may be configured to directly acquire the sensor data from the acquisition unit 231. First, the calculation unit 235 converts the coordinate system of the acquired sensor data from the local coordinate system to the world coordinate system. When the user is standing upright, the local coordinate system (x axis, y axis, z axis) and the world coordinate system (X axis, Y axis, Z axis) coincide. Since the spatial posture of the measurement device 21 changes while the user is walking, the local coordinate system (x axis, y axis, z axis) and the world coordinate system (X axis, Y axis, Z axis) do not match. Therefore, the calculation unit 235 converts the sensor data acquired by the measurement device 21 from the local coordinate system (x axis, y axis, z axis) of the measurement device 21 into the world coordinate system (X axis, Y axis, Z axis). When the gait event can be detected using the sensor data in the local coordinate system, coordinate transformation from the local coordinate system to the world coordinate system may be omitted.

Using the acquired sensor data, the calculation unit 235 generates time-series data of a physical amount relating to the movement of the feet measured along with walking of the user wearing the shoe 200 on which the measurement device 21 is installed. For example, the calculation unit 235 generates time-series data such as a spatial acceleration and a spatial angular velocity. The calculation unit 235 integrates the spatial acceleration and the spatial angular velocity to generate time-series data such as a spatial speed, a spatial angle (plantar angle), and a spatial trajectory. The calculation unit 235 generates time-series data at a predetermined timing or time interval set in accordance with a general gait cycle or a gait cycle unique to the user. The timing at which the calculation unit 235 generates the time-series data can be arbitrarily set. For example, the calculation unit 235 is configured to continue to generate time-series data during a period in which the user keeps walking. For example, the calculation unit 235 may be configured to generate time-series data at a specific timing.

When calculating the gait parameter, the calculation unit 235 extracts time-series sensor data (also referred to as a gait waveform) for one gait cycle from the generated time-series data. Here, the gait waveform does not represent the time-series sensor data as a graph, but is the time-series sensor data itself. For example, the calculation unit 235 detects the timing at the center of the stance phase as the start point of the gait waveform as the start point of the time-series data. For example, the calculation unit 235 may detect the timing of the heel strike or the toe off as the start point of the gait waveform. In a case where the gait parameter is not calculated, the calculation unit 235 may output the generated time-series data to the transmission unit 237.

The calculation unit 235 detects a gait event from the extracted gait waveform for one gait cycle. For example, the calculation unit 235 detects the timing of a characteristic change accompanying the appearance of the gait event in the gait waveform. For example, the calculation unit 235 detects characteristic local maximum and local minimum timings associated with the appearance of the gait event in the gait waveform.

For example, the calculation unit 235 detects gait events such as a heel strike, a toe off, a foot adjacent, a heel rise, a tibia vertical, an opposite toe off, and an opposite heel strike. In the present example embodiment, a state in which the toe is located below the heel (plantarflexion) is defined as positive, and a state in which the toe is located above the heel (dorsiflexion) is defined as negative. In the plantarflexion, the roll angle is maximized at the timing of the toe off. For example, the calculation unit 235 detects the timing at which the roll angle becomes the maximum in the gait waveform of one gait cycle as the timing of the toe off. In dorsiflexion, the roll angle is minimized at the timing of the heel strike. For example, the calculation unit 235 detects the timing at which the roll angle becomes the minimum in the gait waveform of one gait cycle as the timing of the heel strike. For example, the calculation unit 235 detects the timing at the center of the stance phase from the gait waveform of the roll angle. In practice, the timing at which the roll angle indicates the maximum/minimum does not completely match the timing of the toe off/heel strike. Therefore, in the gait waveform for one gait cycle, the gait cycle may be normalized such that the timing at which the roll angle indicates maximum/minimum coincides with the timing of the toe off/heel strike. By normalizing the gait waveform, it is possible to align the timing of appearance of different gait events depending on the person.

The calculation unit 235 calculates a gait parameter based on the detected gait event. For example, the calculation unit 235 calculates the gait parameter using the timings of the detected gait events and the values of the sensor data at the timings of these gait events. For example, the calculation unit 235 calculates the gait parameter for each gait cycle. For example, the calculation unit 235 calculates gait parameters such as a walking speed, a stride length, a grounding angle, a separation angle, a maximum foot-raising height (sensor position), a division (trajectory in the traveling direction), and a toe direction. A description of a method of calculating these gait parameters is omitted. The gait parameter and the sensor data are relevant to the gait data.

The transmission unit 237 acquires the gait data from the calculation unit 235. The transmission unit 237 transmits the acquired gait data to a mobile terminal or the like (not illustrated) on which the estimation device 25 is mounted. For example, the transmission unit 237 transmits the gait data to the mobile terminal via a wire such as a cable. For example, the transmission unit 237 transmits the gait data to a mobile terminal or the like via wireless communication. For example, the transmission unit 237 transmits the gait data to the mobile terminal via a wireless communication function (not illustrated) conforming to a standard such as Bluetooth (registered trademark) or WiFi (registered trademark). The communication function of the transmission unit 237 may conform to a standard other than Bluetooth (registered trademark) or WiFi (registered trademark).

For example, the measurement unit 23 is achieved by a microcomputer or a microcontroller. For example, the measurement unit 23 includes a control circuit and a storage circuit. For example, the control circuit is achieved by a central processing unit (CPU). The storage circuit is implemented by, for example, a volatile memory such as a random access memory (RAM). For example, the storage circuit is achieved by a non-volatile memory such as a read only memory or an electrically erasable and programmable read only memory (EEPROM).

### [Estimation Device]

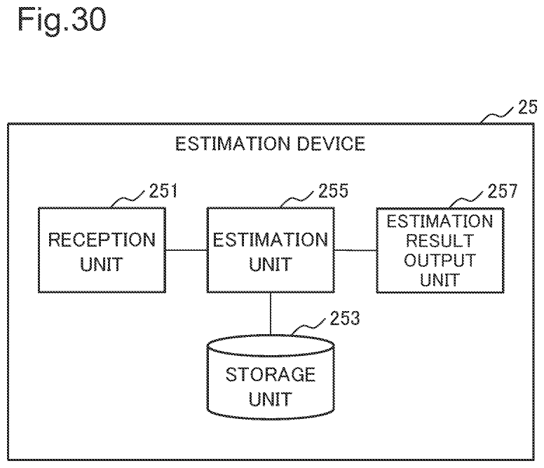

Next, a detailed configuration of the estimation device 25 included in the estimation system 20 will be described with reference to the drawings. FIG. 30 is a block diagram illustrating an example of a configuration of an estimation device 25. The estimation device 25 includes a reception unit 251, a storage unit 253, an estimation unit 255, and an estimation result output unit 257. For example, the estimation device 25 is mounted on a mobile terminal (not illustrated) carried by the user. For example, the estimation device 25 may be achieved by application software or the like installed in a mobile terminal (not illustrated) carried by a user.

The reception unit 251 receives the gait data such as the gait parameter and the sensor data from the measurement device 21. The reception unit 251 outputs the received gait data to the estimation unit 255. The reception unit 251 may store the received gait data in the storage unit 253. For example, the reception unit 251 receives the gait data from the measurement device 21 via wireless communication. For example, the reception unit 251 is configured to receive the gait data from the measurement device 21 via a wireless communication function (not illustrated) conforming to a standard such as Bluetooth (registered trademark) or WiFi (registered trademark). The communication function of the reception unit 251 may conform to a standard other than Bluetooth (registered trademark) or WiFi (registered trademark). For example, the reception unit 251 may receive the gait data from the measurement device 21 via a wire such as a cable.

The storage unit 253 stores the estimation model generated by the learning device 15 of the first example embodiment. The estimation model stored in the storage unit 253 is used for estimation by the estimation unit 255. The storage unit 253 may store the data received by the reception unit 251.

The estimation unit 255 acquires the gait data such as the time-series sensor data and the gait parameter from the reception unit 251. The estimation unit 255 extracts a feature amount from the acquired gait data. The estimation unit 255 inputs the extracted feature amount to the estimation model and outputs a result (gait event index) output from the estimation model.

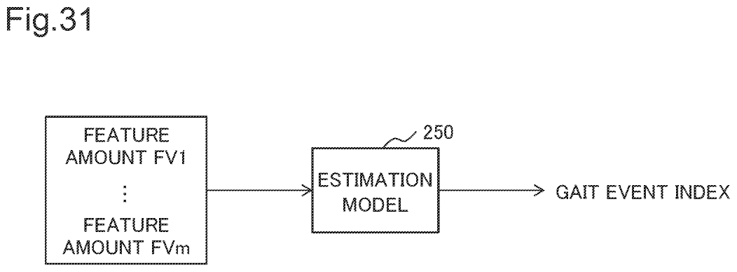

FIG. 31 is a conceptual diagram for explaining an example of estimation by the estimation unit 255. In the example of FIG. 31, estimation is performed using the estimation model 250 generated by the method of the first example embodiment. The estimation model 250 outputs the gait event data (response variable) in response to the input of the feature amount extracted from the gait data based on the sensor data measured by the measurement device 21. The estimation model 250 outputs the gait event data as an estimation result in response to inputs of the feature amounts FV1 to FVm (m is a natural number). For example, in the case of the estimation model 250 that estimates the degree of progression of hallux valgus, the degree of progress according to the input feature amounts FV1 to FVm is output from the estimation model 250. For example, in the case of the estimation model 250 that estimates the degree of pronation/supination of the foot, the degree according to the input feature amounts FV1 to FVm is output from the estimation model 250. As long as the gait event data (response variable) relating to the physical condition can be output as the estimation result in response to the input of the feature amount, the estimation result estimated by the estimation model 250 is not limited.

For example, the estimation unit 255 estimates a score relating to the gait of the user. For example, the score is a numerical value relating to the evaluation of the gait of the user. For example, the estimation unit 255 estimates the physical condition of the user. For example, the physical condition includes the degree of pronation/supination of the foot, the degree of progression of hallux valgus, the degree of progression of knee arthropathy, muscle strength, balance ability, and body flexibility. The estimation processing by the estimation unit 255 is not particularly limited as long as it relates to the gait.

For example, the estimation unit 255 estimates the physical condition in accordance with a numerical value relating to the physical condition such as the center of pressure excursion index CPEI or the HV angle. For example, regarding the center of pressure excursion index CPEI, the estimation device 25 estimates pronation if the index is 9 or less, normal if 9 to 20, and supination if 20 or more. For example, regarding the HV angle, the estimation unit 255 estimates that it is hallux valgus when the HV angle exceeds 20 degrees, estimates that it tends to be hallux valgus when the HV angle exceeds a predetermined threshold less than 20 degrees, and estimates that it is normal when the HV angle is equal to or less than the predetermined threshold. The estimation of the physical condition by the estimation unit 255 is not particularly limited as long as it relates to the gait.

The estimation result output unit 257 acquires the estimation result by the estimation unit 255. The estimation result output unit 257 outputs the estimation result acquired from the estimation unit 255. For example, the estimation result output unit 257 outputs the estimation result by the estimation unit 255 to a display device (not illustrated). For example, the estimation result by the estimation unit 255 is displayed on the screen of the display device. For example, the estimation result by the estimation unit 255 is output to an external system that uses the estimation result. The use of the estimation result by the estimation unit 255 is not particularly limited.

A mobile terminal (not illustrated) on which the estimation device 25 is mountable is a communication device that can be carried by a user. For example, the mobile terminal is a mobile terminal device having a communication function, such as a smartphone, a smart watch, a tablet, or a mobile phone. The mobile terminal receives the gait data from the measurement device 21. The mobile terminal estimates the physical condition of the user using the received gait data. For example, the measurement device 21 displays a result of the data processing of the gait data on the screen of the mobile terminal. For example, a result of data processing of the gait data may be displayed on a screen of a terminal device (not illustrated) visible by the user. For example, the estimation device 25 displays at least one numerical value of the gait data received from the measurement device 21 on the screen of the mobile terminal in real time. For example, the estimation device 25 displays the time-series data of the gait data received from the measurement device 21 on the screen of the mobile terminal in real time. The estimation device 25 may transmit an estimation result using the received gait data to a server, a cloud, or the like. The use of the estimation result estimated by the estimation device 25 is not particularly limited. The estimation device 25 may be installed on a server or a cloud side as long as the gait data measured by the measurement device 21 can be acquired.

### (Operation)

Next, an operation of the estimation system 20 will be described with reference to the drawings. Hereinafter, operations of the measurement device 21 and the estimation device 25 included in the estimation system 20 will be individually described.

### [Measurement Device]

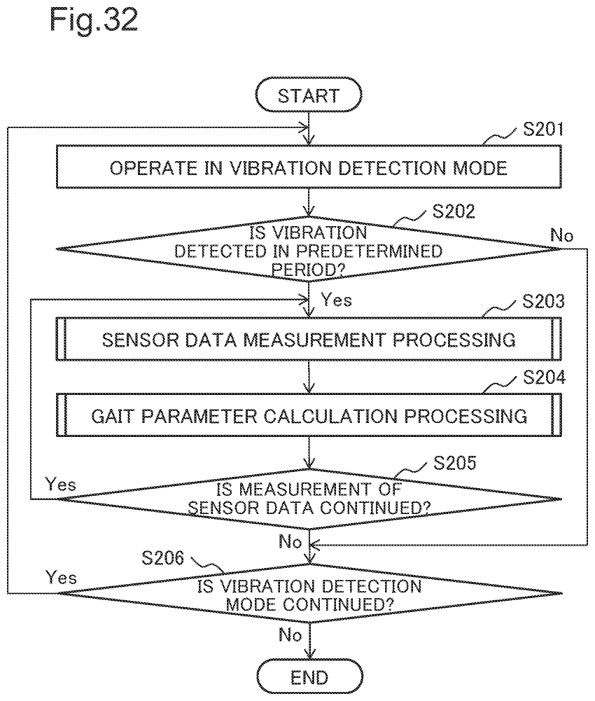

Next, the operation of the measurement device 21 will be described with reference to the drawings. FIG. 32 is a flowchart for explaining an example of the operation of the measurement device 21.

In FIG. 32, the measurement device 21 is operating in a vibration detection mode (step S201). For example, the measurement device 21 is activated in accordance with a user's operation and starts to operate in the vibration detection mode. For example, the measurement device 21 is set to be activated in a preset time zone or timing.

When detecting vibration within the predetermined period (Yes in step S202), the measurement device 21 executes sensor data measurement processing (step S203). In the sensor data measurement processing in step S203, the measurement device 21 measures sensor data. Details of the sensor data measurement processing in step S203 will be described later. When the vibration has not been detected within the predetermined period (No in step S202), the process proceeds to step S206.

After step S203, the measurement device 21 executes gait parameter calculation processing (step S204). In the gait parameter calculation processing of step S204, the measurement device 21 calculates the gait parameter using the sensor data measured in the sensor data measurement processing of step S203. Details of the gait parameter calculation processing in step S204 will be described later.

After step S204, when the measurement of the sensor data is continued (Yes in step S205), the process returns to step S203. The continuation of the measurement of the sensor data may be determined in accordance with a preset condition such as the number of steps and time. If the measurement of the sensor data is not continued (No in step S205) and the vibration detection mode is continued (Yes in step S206), the process returns to step S201. When the vibration detection mode is not continued (No in step S206), the process according to the flowchart of FIG. 32 is ended. The continuation/stop of measurement may be determined in accordance with a predetermined timing, a stop operation of the user, or the like.

### [Sensor Data Measurement Processing]

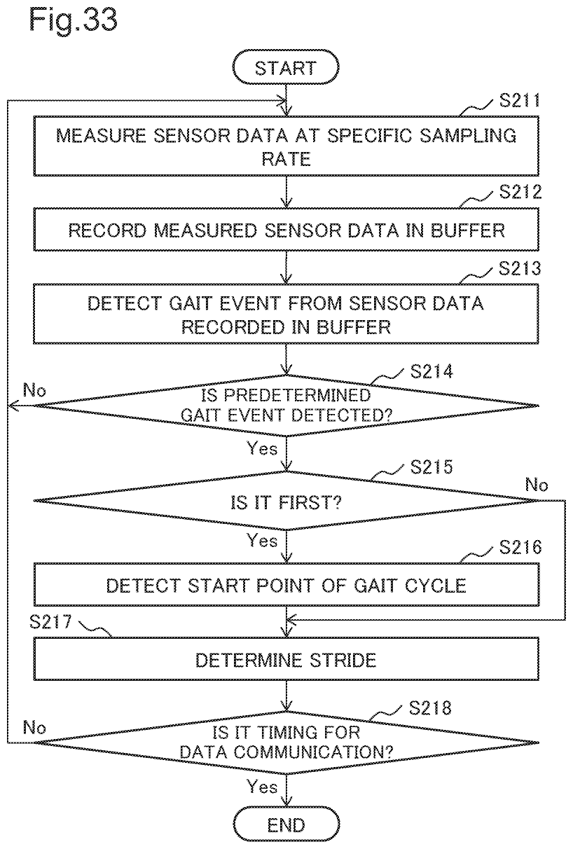

Next, an example of sensor data measurement processing (step S203 in FIG. 32) by the measurement device 21 will be described with reference to the drawings. FIG. 33 is a flowchart for explaining an example of sensor data measurement processing by the measurement device 21. In the description of the processing along the flowchart of FIG. 33, the measurement device 21 will be described as an operation subject.

In FIG. 32, first, the measurement device 21 measures sensor data at a specified sampling rate (step S211). The measurement device 21 measures sensor data such as acceleration and angular velocity.

Next, the measurement device 21 records the acquired sensor data in the buffer (storage unit 233) (step S212).

Next, the measurement device 21 detects a gait event from the sensor data recorded in the buffer (step S213).

When the predetermined gait event is detected (Yes in step S214) and this is the first time (Yes in step S215), the measurement device 21 detects the start point of the gait cycle (step S216). For example, the measurement device 21 detects the heel strike, the toe off, the timing at the center of the stance phase, and the like as start points of the gait cycle. When the gait event is not the first time (No in step S215), the process proceeds to step S217.

After step S216 or in the case of No in step S215, the measurement device 21 performs stride determination (step S217). In the stride determination, the measurement device 21 determines acquisition of sensor data for one step (for one stride).

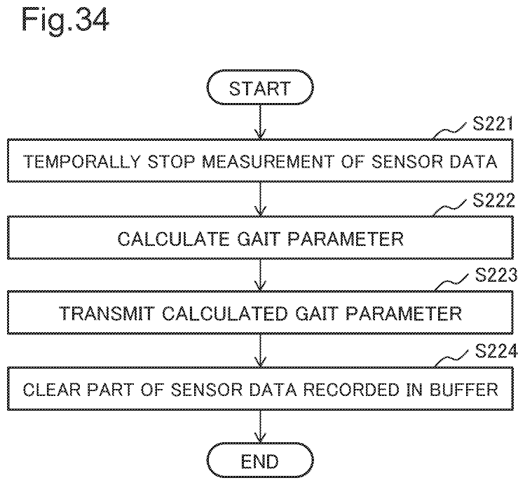

Here, when it is the timing of data communication (Yes in step S218), the process proceeds to step S204 (step S221 in FIG. 34) of the flowchart in FIG. 32. For example, the timing of data communication is set to the swing phase. When it is not the timing of data communication (No in step S218), the process returns to step S211.

### [Gait Parameter Calculation Processing]

Next, an example of the gait parameter calculation processing (step S204 in FIG. 32) by the measurement device 21 will be described with reference to the drawings. FIG. 34 is a flowchart for explaining an example of the gait parameter calculation processing by the measurement device 21. In the description of the processing along the flowchart of FIG. 34, the measurement device 21 will be described as an operation subject.

In FIG. 32, first, the measurement device 21 temporarily stops the measurement of the sensor data (step S221). In the case of a single-tasking microcomputer, the sensor data measurement and the gait parameter calculation cannot be performed at the same time, and thus the sensor data measurement is temporarily stopped. In the case of a multi-tasking microcomputer, since the sensor data measurement and the gait parameter calculation can be performed simultaneously, step S221 may be omitted.

Next, the measurement device 21 calculates the gait parameter using the sensor data stored in the buffer (storage unit 233) (step S222). For example, the measurement device 21 calculates gait parameters such as a walking speed, a stride length, a grounding angle, a separation angle, a maximum foot-raising height (sensor position), a division (trajectory in the traveling direction), and a toe direction. In a case where the sensor data itself is transmitted without calculating the gait parameter, step S222 is omitted.

Next, the measurement device 21 transmits the calculated gait parameter (gait data) (step S223). For example, the measurement device 21 transmits gait parameters such as a walking speed, a stride length, a grounding angle, a separation angle, a maximum foot-raising height (sensor position), a division (trajectory in the traveling direction), and a toe direction. For example, the measurement device 21 may transmit the sensor data itself.

Next, the measurement device 21 clears a part of the sensor data stored in the buffer (storage unit 233) (step S224). For example, the measurement device 21 deletes the sensor data used to calculate the transmitted gait parameter from the buffer (storage unit 233). After step S224, the process proceeds to step S205 in the flowchart of FIG. 32.

### [Estimation Device]

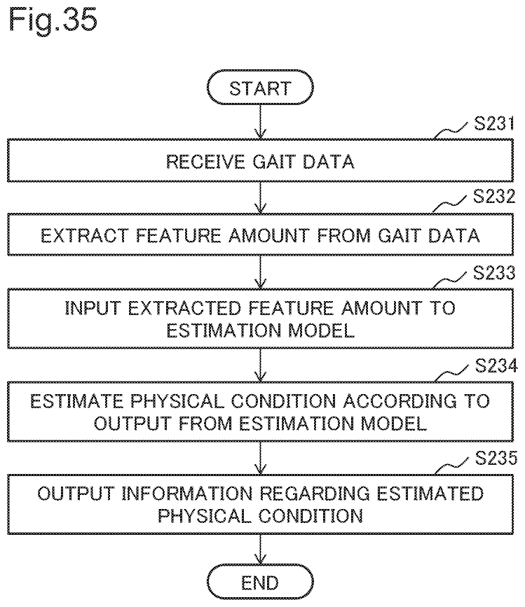

Next, details of the estimation device 25 will be described with reference to the drawings. FIG. 35 is a flowchart for explaining an example of the operation of the estimation device 25. In the description along the flowchart of FIG. 35, the estimation device 25 will be described as an operation subject.

In FIG. 35, first, the estimation device 25 receives the gait data from the measurement device 21 (step S231). The estimation device 25 receives the gait data such as the sensor data and the gait parameter.

Next, the estimation device 25 extracts a feature amount from the received gait data (step S232).

Next, the estimation device 25 inputs the extracted feature amount to the estimation model (step S233). The estimation model outputs a response variable (gait event index) in response to the input of the feature amount. In the case of an estimation model that outputs a response variable in response to an input of gait data such as sensor data and a gait parameter, the gait data is input. For example, the estimation model outputs a score relating to the gait of the user as a response variable. For example, the estimation model outputs a numerical value relating to the gait parameter as a response variable. For example, the estimation device 25 outputs numerical values relating to the degree of pronation/supination of the foot, the degree of progression of hallux valgus, the degree of progression of knee arthropathy, muscle strength, balance ability, and body flexibility, and the like as response variables. For example, the estimation model outputs numerical values such as the center of pressure excursion index (CPEI) and the hallux valgus (HV) angle as response variables.

Next, the estimation device 25 estimates the physical condition in accordance with the output from the estimation model (step S234). The estimation device 25 estimates the physical condition in accordance with the response variable output from the estimation model. For example, the estimation device 25 estimates a score relating to the gait of the user. For example, the estimation device 25 estimates the physical condition in accordance with the numerical value relating to the gait parameter. For example, the estimation device 25 estimates the physical condition in accordance with numerical values relating to the degree of pronation/supination of the foot, the degree of progression of hallux valgus, the degree of progression of knee arthropathy, muscle strength, balance ability, flexibility of the body, and the like.

Next, the estimation device 25 outputs information relating to the estimated physical condition (step S235). For example, the estimation device 25 outputs the estimation result to a display device (not illustrated). For example, the estimation result by the estimation device 25 is displayed on the screen of the display device. For example, the estimation result by the estimation device 25 is output to a system that uses the estimation result. The use of the estimation result by the estimation device 25 is not particularly limited.

### APPLICATION EXAMPLE

Next, an application example of the present example embodiment will be described with reference to the drawings. In the application example of the present example embodiment, the measurement device is installed in the shoe of the user, and the gait data measured by the measurement device is transmitted to the mobile terminal possessed by the user. The sensor data transmitted to the mobile terminal is processed by an estimation device implemented in the mobile terminal. For example, the function of the estimation device is provided as an application that can be installed in a mobile terminal.

### Application Example 2-1

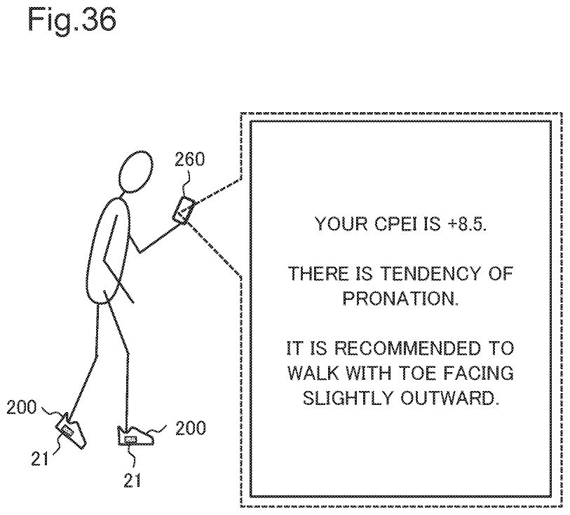

FIG. 36 is a conceptual diagram for explaining Application Example 2-1. In the present application example, information according to the estimation result by the estimation device 25 is displayed on the screen of a mobile terminal 260 of the user wearing the shoe 200 on which the measurement device 21 is installed.

In the example of FIG. 36, an estimation result of “Your CPEI is +8.5” is displayed on the screen of the mobile terminal 260 carried by the user. Information relating to the physical condition according to the estimation result, such as “There is a tendency of pronation.”, is displayed on the screen of the mobile terminal 260. Furthermore, recommendation information of “It is recommended to walk with the toe facing slightly outward.” is displayed on the screen of the mobile terminal 260 in accordance with the physical condition. The user who has browsed the information displayed on the screen of the mobile terminal 260 can take an action according to the information. For example, the user who has browsed the information displayed on the screen of the mobile terminal 260 can exercise or walk in a manner suitable for the user in accordance with the information. For example, the user who has browsed the information displayed on the screen of the mobile terminal 260 can contact a medical institution or the like regarding his/her physical condition in accordance with the information.

The information according to the estimation result by the estimation device 25 is not limited to the screen of the mobile terminal 260 as long as it is a screen visually recognizable by the user, and may be displayed on a screen of a stationary personal computer or a dedicated terminal. The information according to the estimation result by the estimation device 25 may be an image according to the estimation result instead of character information. The information according to the estimation result by the estimation device 25 may be notified in a preset pattern such as sound or vibration.

### Application Example 2-2

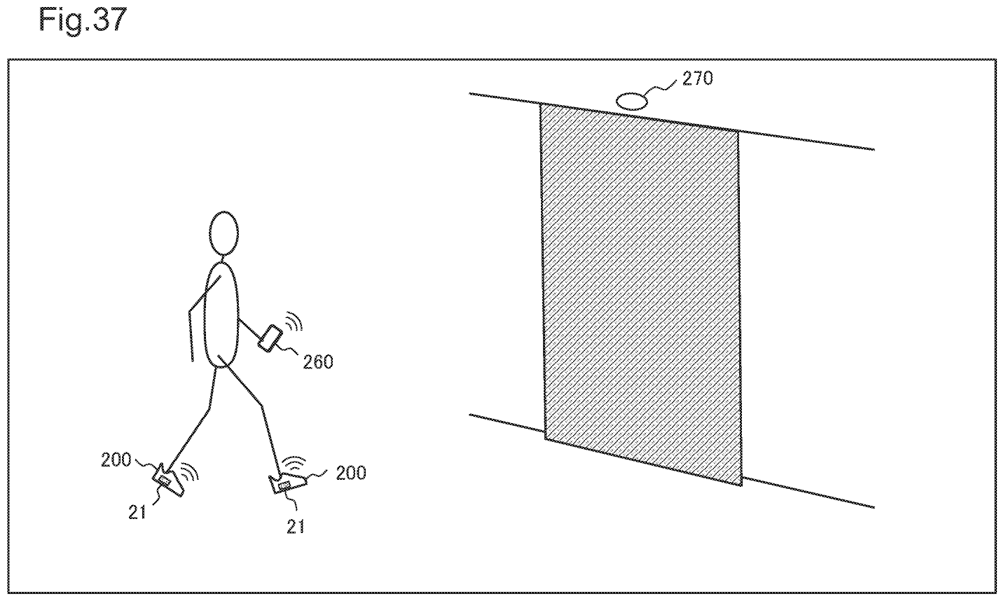

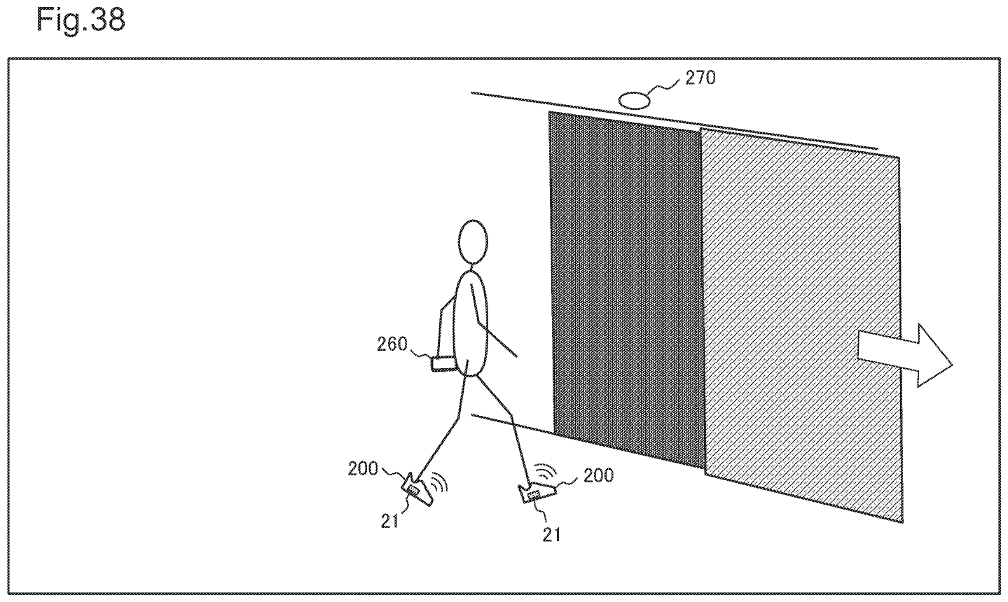

FIGS. 37 and 38 are conceptual diagrams for explaining Application Example 2-2. In the present application example, the user is authenticated in accordance with the authentication information transmitted from the mobile terminal 260 of the user wearing the shoe 200 on which the measurement device 21 is installed. In a case where the personal authentication according to the gait data is performed, the pair data including the already accumulated gait data may be extended by the method of the first example embodiment. The identification number is invariable because it is unique to an individual, but can be regarded as a variable.

FIG. 37 illustrates a state in which the user wearing the shoe 200 on which the measurement device 21 is installed approaches a door that requires authentication. An opening and closing control device 270 that receives an identification number (also referred to as authentication information) estimated by the estimation device 25 and controls opening and closing of the door in accordance with the received identification number is installed above the door. The opening and closing control device 270 controls a driving device (not illustrated) that opens and closes the door in accordance with the received identification number. The estimation device 25 mounted on the mobile terminal 260 estimates the identification number of the user using the feature amount extracted from the gait data measured by the measurement device 21. The estimation device 25 transmits the estimated identification number. The identification number transmitted from the estimation device 25 is received by the opening and closing control device 270. The identification number received by the opening and closing control device 270 is authenticated by the opening and closing control device 270, an authentication system mounted on a server or the like connected to the opening and closing control device 270, or the like. When the identification number is an authorized number, the opening and closing control device 270 opens the door. When the identification number is not an authorized number, the opening and closing control device 270 does not open the door.

FIG. 38 illustrates a state in which the door is opened by the opening and closing control device 270 in accordance with the identification number transmitted from the estimation device 25. The user wearing the shoe 200 on which the measurement device 21 is installed can enter the inside of a building or the like through the opened door.

For example, in a case where a person B different from the user walks wearing shoes 200 on which the measurement device 21 is installed, the identification number is estimated in accordance with the feature amount extracted from the gait data of the person B. When the identification number of the person B is an authorized number, the opening and closing control device 270 opens the door. When the identification number of the person B is not an authorized number, the opening and closing control device 270 does not open the door. For example, in a case where the shoe 200 on which the measurement device 21 is installed is worn by a person other than the user, the door is not opened unless the identification number estimated in accordance with the gait data is an authorized number.

For example, authentication using an identification number estimated in accordance with the gait data and other authentication may be combined. For example, in a case where both the identification number of the shoe 200 or the measurement device 21 and the identification number estimated in accordance with the gait data are authenticated, the opening and closing control device 270 can be configured to open the door. With this configuration, a person other than the user cannot pass through the door by wearing the shoes 200, and thus security is improved. For example, authentication by an identification number according to the gait data may be combined with authentication such as face authentication, fingerprint authentication, palm print authentication, or vein authentication.

A variable estimated in accordance with the gait data may be used as the identification number. For example, when the variable estimated in accordance with the gait data is a value within a predetermined range from the authorized identification number, authentication can be performed. With this configuration, even the entry of a user who is permitted to enter can be restricted in accordance with the variation in the gait data. For example, in a situation in which a user having a slight cold shows a different gait from usual, and the estimated variable is out of the predetermined range from the permitted identification number, the entry of the user can be restricted. For example, in accordance with the gait of the user, display information for recommending visit to a health management center at work or recommending recuperation at home may be displayed near the door or on the screen of the mobile terminal 260. For example, in a place where the user has to slowly walk through, the entry of the user who is about to run through can be restricted. For example, display information for recommending slow walking or imposing a penalty for running may be displayed near the door or on the screen of the mobile terminal 260 in accordance with the gait of the user.

As described above, the estimation system of the present example embodiment includes the measurement device and the estimation device. The measurement device is disposed on the user's footwear. The measurement device measures a spatial acceleration and a spatial angular velocity in accordance with walking of the user, and generates sensor data based on the measured spatial acceleration and spatial angular velocity. The measurement device generates measurement gait data using the generated sensor data. The measurement device transmits the generated measurement gait data to the estimation device. The estimation device includes a reception unit, a storage unit, an estimation unit, and an estimation result output unit. The reception unit receives the measurement gait data derived using the sensor data relating to the movement of the user's feet. The storage unit stores the estimation model generated by the learning system of the first example embodiment. The estimation unit inputs the received measurement gait data to the estimation model. The estimation unit estimates the physical condition of the user in accordance with the response variable output from the estimation model. The estimation result output unit outputs information relating to the estimated physical condition of the user.

The estimation system of the present example embodiment estimates the physical condition of the user using the estimation model generated by the learning system of the first example embodiment. Since the estimation model generated by the learning system of the first example embodiment is generated using the extended dataset, accuracy and versatility are high. Therefore, according to the estimation system of the present example embodiment, estimation with high accuracy and versatility can be executed.

In an aspect of the present example embodiment, the estimation result output unit displays the estimated information relating to the physical condition of the user on a screen of a mobile terminal carried by the user. According to the present aspect, the user himself/herself can confirm the estimation result estimated in accordance with the gait of the user in real time.

In an aspect of the present example embodiment, the storage unit stores an estimation model that outputs a response variable relating to an identification number in response to an input of measurement gait data. The estimation unit estimates the identification number of the user in accordance with the response variable output from the estimation model in response to the input of the measurement gait data. The output unit transmits the estimated identification number to an authentication device that performs authentication using the identification number. According to the present aspect, authentication according to the gait can be achieved.

### Third Example Embodiment

Next, a data generation device according to a third example embodiment will be described with reference to the drawings. The data generation device of the present example embodiment has a simplified configuration of the data generation device of the first example embodiment.

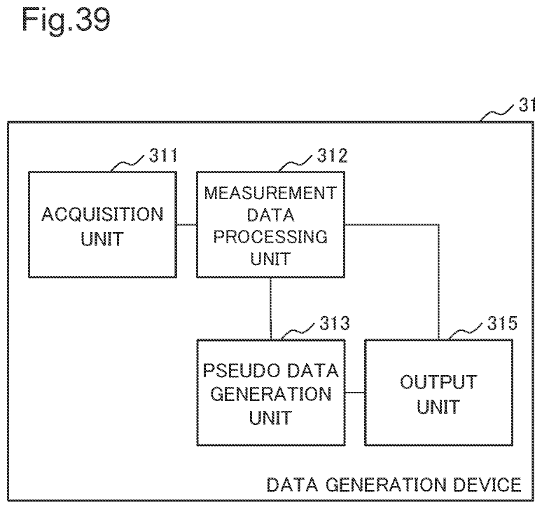

FIG. 39 is a block diagram illustrating an example of a configuration of a data generation device 31 according to the present example embodiment. The data generation device 31 includes an acquisition unit 311, a measurement data processing unit 312, a pseudo data generation unit 313, and an output unit 315.

The acquisition unit 311 acquires pair data in which measurement gait data relating to sensor data measured in accordance with the movement of a user's feet and a response variable relevant to the measurement gait data are combined. The measurement data processing unit 312 generates a measurement dataset vector by combining a feature amount vector calculated using a feature amount extracted from the measurement gait data and a response variable. The measurement data processing unit 312 generates a covariance matrix relating to a plurality of pieces of pair data. The pseudo data generation unit 313 generates pseudo gait data using the measurement gait data. The pseudo data generation unit 313 generates a pseudo dataset vector by combining a pseudo feature amount vector calculated using a pseudo feature amount extracted from the pseudo gait data and a pseudo response variable generated using a covariance matrix relating to the pseudo feature amount vector. The output unit 315 outputs a dataset including the measurement dataset vector and the pseudo dataset vector.

As described above, the data generation device of the present example embodiment generates the pseudo dataset vector by combining the pseudo feature amount vector calculated using the pseudo feature amount extracted from the pseudo gait data and the pseudo response variable generated using the covariance matrix relating to the plurality of pieces of pair data. Therefore, according to the data generation device of the present example embodiment, the dataset used for learning can be generated even if the response variable is a continuous value.

### (Hardware)

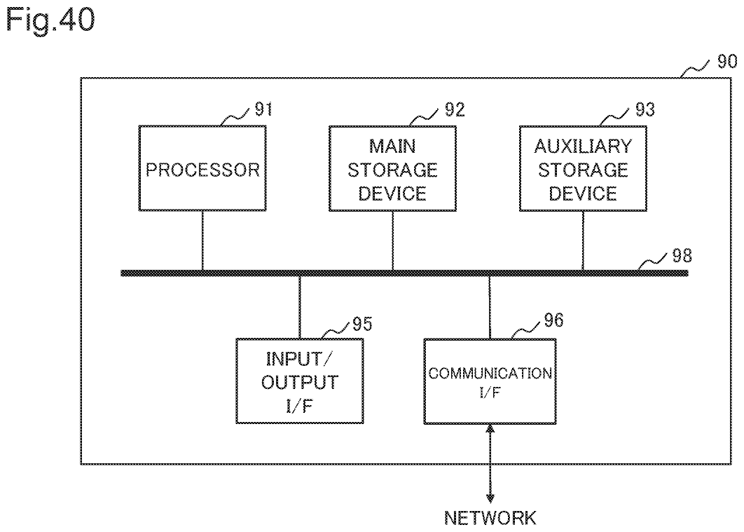

Here, a hardware configuration for executing processing of the control unit according to each example embodiment of the present disclosure will be described using an information processing device 90 of FIG. 40 as an example. The information processing device 90 in FIG. 40 is a configuration example for executing the control and processing of each example embodiment, and does not limit the scope of the present disclosure.

As illustrated in FIG. 40, the information processing device 90 includes a processor 91, a main storage device 92, an auxiliary storage device 93, an input/output interface 95, and a communication interface 96. In FIG. 40, the interface is abbreviated as an I/F. The processor 91, the main storage device 92, the auxiliary storage device 93, the input/output interface 95, and the communication interface 96 are data-communicably connected to each other via a bus 98. The processor 91, the main storage device 92, the auxiliary storage device 93, and the input/output interface 95 are connected to a network such as the Internet or an intranet via communication interface 96.

The processor 91 develops a program stored in the auxiliary storage device 93 or the like in the main storage device 92. The processor 91 executes the program developed in the main storage device 92. In the present example embodiment, a software program installed in the information processing device 90 may be used. The processor 91 executes processing or control according to each example embodiment.

The main storage device 92 has a region in which a program is developed. A program stored in the auxiliary storage device 93 or the like is developed in the main storage device 92 by the processor 91. The main storage device 92 is implemented by, for example, a volatile memory such as a dynamic random access memory (DRAM). A nonvolatile memory such as a magnetoresistive random access memory (MRAM) may be configured and added as the main storage device 92.

The auxiliary storage device 93 stores various data such as programs. The auxiliary storage device 93 is implemented by a local disk such as a hard disk or a flash memory. Various data may be stored in the main storage device 92, and the auxiliary storage device 93 may be omitted.

The input/output interface 95 is an interface for connecting the information processing device 90 and a peripheral device. The communication interface 96 is an interface for connecting to an external system or device through a network such as the Internet or an intranet based on a standard or a specification. The input/output interface 95 and the communication interface 96 may be shared as an interface connected to an external device.

An input device such as a keyboard, a mouse, or a touch panel may be connected to the information processing device 90 as necessary. These input devices are used to input information and settings. When the touch panel is used as an input device, the display screen of the display device may also serve as the interface of the input device. Data communication between the processor 91 and the input device may be mediated by the input/output interface 95.

The information processing device 90 may be provided with a display device for displaying information. In a case where a display device is provided, the information processing device 90 may include a display control device (not illustrated) for controlling display of the display device. The display device may be connected to the information processing device 90 via input/output interface 95.

The information processing device 90 may be provided with a drive device. The drive device mediates reading of data and a program from a recording medium, writing of a processing result of the information processing device 90 to the recording medium, and the like between the processor 91 and the recording medium (program recording medium). The drive device may be connected to the information processing device 90 via input/output interface 95.

The above is an example of the hardware configuration for enabling the control and processing according to each example embodiment of the present invention. The hardware configuration of FIG. 40 is an example of a hardware configuration for executing the control and processing of each example embodiment, and does not limit the scope of the present invention. A program for causing a computer to execute the control and processing according to each example embodiment is also included in the scope of the present invention. Further, a program recording medium in which the program according to each example embodiment is recorded is also included in the scope of the present invention. The recording medium can be achieved by, for example, an optical recording medium such as a compact disc (CD) or a digital versatile disc (DVD). The recording medium may be implemented by a semiconductor recording medium such as a universal serial bus (USB) memory or a secure digital (SD) card. The recording medium may be implemented by a magnetic recording medium such as a flexible disk, or another recording medium. When a program executed by the processor is recorded in a recording medium, the recording medium is associated to a program recording medium.

The components of each example embodiment may be arbitrarily combined. The components of each example embodiment may be implemented by software or may be implemented by a circuit.

Although the present invention has been described with reference to the example embodiments, the present invention is not limited to the above example embodiments. Various modifications that can be understood by those skilled in the art can be made to the configuration and details of the present invention within the scope of the present invention.

Some or all of the above example embodiments may be described as the following Supplementary Notes, but are not limited to the following.

### (Supplementary Note 1)

A data generation device including:
an acquisition unit that acquires pair data by combing measurement gait data relating to sensor data measured in accordance with the movement of a user's feet and a response variable relevant to the measurement gait data;
a measurement data processing unit that generates a measurement dataset vector by combining the response variable and a feature amount vector calculated using a feature amount extracted from the measurement gait data, and generates a covariance matrix relating to a plurality of pieces of the pair data;
a pseudo data generation unit that generates pseudo gait data using the measurement gait data, and generates a pseudo dataset vector by combining a pseudo feature amount vector calculated using a pseudo feature amount extracted from the pseudo gait data and a pseudo response variable generated using a covariance matrix relating to the pseudo feature amount vector; and
an output unit that outputs the dataset including the measurement dataset vector and the pseudo dataset vector.

### (Supplementary Note 2)

The data generation device according to Supplementary Note 1, in which
the pseudo data generation unit is configured to:
add fluctuation to a plurality of pieces of the measurement gait data included in a plurality of pieces of the pair data to generate a plurality of pieces of the pseudo gait data.

### (Supplementary Note 3)

The data generation device according to Supplementary Note 1 or 2, in which
the pseudo data generation unit is configured to:
add noise to a plurality of pieces of the measurement gait data included in a plurality of pieces of the pair data to generate a plurality of pieces of the pseudo gait data.

### (Supplementary Note 4)

The data generation device according to any one of Supplementary Notes 1 to 3, in which
the pseudo data generation unit is configured to:
generate a plurality of pieces of the pseudo gait data using the measurement gait data;
extract at least one of the pseudo feature amounts from the generated pseudo gait data; and
generate the pseudo feature amount vector for each piece of the pseudo gait data using the pseudo feature amount extracted from the pseudo gait data.

### (Supplementary Note 5)

The data generation device according to any one of Supplementary Notes 1 to 4, in which
the measurement data processing unit is configured to:
extract at least one of the feature amounts from the measurement gait data derived using the sensor data for each gait cycle;
generate the feature amount vector for each piece of measurement gait data using the feature amount extracted from the measurement gait data for each gait cycle; and
add the response variable associated with the measurement gait data to an end of the feature amount vector generated for each piece of the measurement gait data to generate the measurement dataset vector for each piece of the measurement gait data.

### (Supplementary Note 6)

The data generation device according to any one of Supplementary Notes 1 to 5, wherein
the measurement data processing unit is configured to:
calculate an average vector of a plurality of the feature amount vectors calculated from a plurality of pieces of the measurement gait data with respect to a plurality of pieces of the pair data:
generate the covariance matrix with respect to a plurality of the measurement dataset vectors generated for each piece of the measurement gait data;
derive an upper triangular matrix of the covariance matrix by performing Cholesky decomposition on the generated covariance matrix; and
calculate an average value of a plurality of the response variables for a plurality of pieces of the pair data, and
the pseudo data generation unit is configured to:
subtract the average vector of the feature amount from each of a plurality of pieces of the pseudo gait data to calculate a pseudo deviation vector of each of a plurality of pieces of the pseudo gait data:
generate a pseudo variance vector by assigning a random value from 0 to 1 to an end of each of a plurality of the calculated pseudo deviation vectors:
integrate a column at an end of the upper triangular matrix to each of a plurality of the generated pseudo variance vectors, and calculate a deviation of the pseudo response variable for each piece of the pseudo gait data; and
calculate the pseudo response variable relevant to the pseudo gait data by adding a deviation of the pseudo response variable calculated for each piece of the pseudo gait data to an average value of the response variables.

### (Supplementary Note 7)

The data generation device according to any one of Supplementary Notes 1 to 6, in which
the output unit is configured to:
display information relating to the generated dataset on a screen of a terminal device.

### (Supplementary Note 8)

A learning system including:
the data generation device according to any one of Supplementary Notes 1 to 7; and
a learning device that acquires a dataset output from the data generation device and generates an estimation model that outputs a response variable according to a physical condition of a user in response to an input of measurement gait data using the acquired dataset.

### (Supplementary Note 9)

An estimation system including:
a storage unit that stores an estimation model generated by the learning system according to Supplementary Note 8;
a reception unit that receives measurement gait data derived using sensor data relating to a movement of a user's feet;
an estimation unit that inputs the received measurement gait data to the estimation model and estimates a physical condition of the user in accordance with a response variable output from the estimation model; and
an estimation result output unit that outputs information relating to the estimated physical condition of the user.

### (Supplementary Note 10)

The estimation system according to Supplementary Note 9, in which
the estimation result output unit is configured to:
display information relating to the estimated physical condition of the user on a screen of a mobile terminal carried by the user.

### (Supplementary Note 11)

The estimation system according to Supplementary Note 9 or 10, in which
the storage unit is configured to:
store the estimation model that outputs the response variable relating to an identification number in response to an input of the measurement gait data,
the estimation unit is configured to:
estimate the identification number of the user in accordance with the response variable output from the estimation model in response to an input of the measurement gait data, and
the output unit is configured to:
transmit the estimated identification number to an authentication device that performs authentication using the identification number.

### (Supplementary Note 12)

The estimation system according to any one of Supplementary Notes 9 to 11, including: a measurement device that is disposed on footwear of the user, measures a spatial acceleration and a spatial angular velocity in accordance with walking of the user, generates the sensor data based on the measured spatial acceleration and the measured spatial angular velocity, generates the measurement gait data using the generated sensor data, and transmits the generated measurement gait data to the reception unit.

### (Supplementary Note 13)

A data generation method causing a computer to execute:
acquiring pair data in which measurement gait data relating to sensor data measured in accordance with a movement of a user's feet and a response variable relevant to the measurement gait data are combined:
generating a measurement dataset vector by combining the response variable and a feature amount vector calculated using a feature amount extracted from the measurement gait data:
generating a covariance matrix for a plurality of pieces of the pair data;
generating pseudo gait data using the measurement gait data:
generating a pseudo dataset vector by combining a pseudo feature amount vector calculated using a pseudo feature amount extracted from the pseudo gait data and a pseudo response variable generated using the covariance matrix relating to the pseudo feature amount vector; and
outputting a dataset including the measurement dataset vector and the pseudo dataset vector.

### (Supplementary Note 14)

A program causing a computer to execute:
processing of acquiring pair data by combing measurement gait data relating to sensor data measured in accordance with the movement of a user's feet and a response variable relevant to the measurement gait data;
processing of generating a measurement dataset vector by combining the response variable and a feature amount vector calculated using a feature amount extracted from the measurement gait data;
processing of generating a covariance matrix relating to a plurality of pieces of the pair data;
processing of generating pseudo gait data using the measurement gait data;
processing of generating a pseudo dataset vector by combining a pseudo feature amount vector calculated using a pseudo feature amount extracted from the pseudo gait data and a pseudo response variable generated using the covariance matrix relating to the pseudo feature amount vector; and
processing of outputting a dataset including the measurement dataset vector and the pseudo dataset vector.

### REFERENCE SIGNS LIST

10 learning system
11, 31 data generation device
15 learning device
20 estimation system
21 measurement device
22 sensor
23 measurement unit
25 estimation device
110 measurement device
111, 311 acquisition unit
112, 312 measurement data processing unit
113, 313 pseudo data generation unit
115, 315 output unit
151 dataset acquisition unit
153 learning unit
155 storage unit
221 acceleration sensor
222 angular velocity sensor
231 acquisition unit
233 storage unit
235 calculation unit
237 transmission unit
250 estimation model
251 reception unit
253 storage unit
255 estimation unit
257 estimation result output unit

What is claimed is:

1. A data generation device comprising:
a first memory storing instructions; and
a first processor connected to the first memory and configured to execute the instructions to:
acquire pair data by combing measurement gait data relating to sensor data measured in accordance with the movement of a user's feet and a response variable relevant to the measurement gait data;
generate a measurement dataset vector by combining the response variable and a feature amount vector calculated using a feature amount extracted from the measurement gait data, and generate a covariance matrix relating to a plurality of pieces of the pair data;
generate pseudo gait data using the measurement gait data, and generate a pseudo dataset vector by combining a pseudo feature amount vector calculated using a pseudo feature amount extracted from the pseudo gait data and a pseudo response variable generated using a covariance matrix relating to the pseudo feature amount vector; and
output the dataset including the measurement dataset vector and the pseudo dataset vector.

2. The data generation device according to claim 1, wherein
the first processor is configured to execute the instructions to
add fluctuation to a plurality of pieces of the measurement gait data included in a plurality of pieces of the pair data to generate a plurality of pieces of the pseudo gait data.

3. The data generation device according to claim 1, wherein
the first processor is configured to execute the instructions to
add noise to a plurality of pieces of the measurement gait data included in a plurality of pieces of the pair data to generate a plurality of pieces of the pseudo gait data.

4. The data generation device according to claim 1, wherein
the first processor is configured to execute the instructions to
generate a plurality of pieces of the pseudo gait data using the measurement gait data,
extract at least one of the pseudo feature amounts from the generated pseudo gait data, and
generate the pseudo feature amount vector for each piece of the pseudo gait data using the pseudo feature amount extracted from the pseudo gait data.

5. The data generation device according to claim 1, wherein
the first processor is configured to execute the instructions to
extract at least one of the feature amounts from the measurement gait data derived using the sensor data for each gait cycle,
generate the feature amount vector for each piece of the measurement gait data using the feature amount extracted from the measurement gait data for each gait cycle, and
add the response variable associated with the measurement gait data to an end of the feature amount vector generated for each piece of the measurement gait data to generate the measurement dataset vector for each piece of the measurement gait data.

6. The data generation device according to claim 1, wherein
the first processor is configured to execute the instructions to
calculate an average vector of a plurality of the feature amount vectors calculated from a plurality of pieces of the measurement gait data with respect to a plurality of pieces of the pair data,
generate the covariance matrix with respect to a plurality of the measurement dataset vectors generated for each piece of the measurement gait data,
derive an upper triangular matrix of the covariance matrix by performing Cholesky decomposition on the generated covariance matrix, and
calculate an average value of a plurality of the response variables for a plurality of pieces of the pair data,
subtract the average vector of the feature amount from each of a plurality of pieces of the pseudo gait data to calculate a pseudo deviation vector of each of a plurality of pieces of the pseudo gait data,
generate a pseudo variance vector by assigning a random value from 0 to 1 to an end of each of a plurality of the calculated pseudo deviation vectors,
integrate a column at an end of the upper triangular matrix to each of a plurality of the generated pseudo variance vectors, and calculate a deviation of the pseudo response variable for each piece of the pseudo gait data, and
calculate the pseudo response variable relevant to the pseudo gait data by adding a deviation of the pseudo response variable calculated for each piece of the pseudo gait data to an average value of the response variables.

7. The data generation device according to claim 1, wherein
the first processor is configured to execute the instructions to
display information relating to the generated dataset on a screen of a terminal device.

8. A learning system comprising:
the data generation device according to claim 1 and
a learning device comprising
a second memory storing instructions; and
a second processor connected to the second memory and configured to execute the instructions to
acquire a dataset output from the data generation device, and
generate an estimation model that outputs a response variable according to a physical condition of a user in response to an input of measurement gait data using the acquired dataset.

9. An estimation system comprising:
a storage configured to store an estimation model generated by the learning system according to claim 8;
a third memory storing instructions; and
a third processor connected to the third memory and configured to execute the instructions to
receive measurement gait data derived using sensor data relating to a movement of a user's feet;
input the received measurement gait data to the estimation model;
estimate a physical condition of the user in accordance with a response variable output from the estimation model; and
output information relating to the estimated physical condition of the user.

10. The estimation system according to claim 9, wherein
the third processor is configured to execute the instructions to
display information relating to the estimated physical condition of the user on a screen of a mobile terminal carried by the user.

11. The estimation system according to claim 9, wherein
the storage is configured to
store the estimation model that outputs the response variable relating to an identification number in response to an input of the measurement gait data,
the third processor is configured to execute the instructions to
estimate the identification number of the user in accordance with the response variable output from the estimation model in response to an input of the measurement gait data, and
transmit the estimated identification number to an authentication device that performs authentication using the identification number.

12. The estimation system according to claim 9, further comprising
a measurement device that is disposed on footwear of the user, measures a spatial acceleration and a spatial angular velocity in accordance with walking of the user, generates the sensor data based on the measured spatial acceleration and the measured spatial angular velocity, generates the measurement gait data using the generated sensor data, and transmits the generated measurement gait data to the reception means.

13. A data generation method causing a computer to execute:
acquiring pair data in which measurement gait data relating to sensor data measured in accordance with a movement of a user's feet and a response variable relevant to the measurement gait data are combined;
generating a measurement dataset vector by combining the response variable and a feature amount vector calculated using a feature amount extracted from the measurement gait data;
generating a covariance matrix for a plurality of pieces of the pair data;
generating pseudo gait data using the measurement gait data;
generating a pseudo dataset vector by combining a pseudo feature amount vector calculated using a pseudo feature amount extracted from the pseudo gait data and a pseudo response variable generated using the covariance matrix relating to the pseudo feature amount vector; and
outputting a dataset including the measurement dataset vector and the pseudo dataset vector.

14. A non-transitory recording medium having stored therein a program causing a computer to execute:
processing of acquiring pair data by combing measurement gait data relating to sensor data measured in accordance with the movement of a user's feet and a response variable relevant to the measurement gait data;
processing of generating a measurement dataset vector by combining the response variable and a feature amount vector calculated using a feature amount extracted from the measurement gait data;
processing of generating a covariance matrix relating to a plurality of pieces of the pair data;
processing of generating pseudo gait data using the measurement gait data;
processing of generating a pseudo dataset vector by combining a pseudo feature amount vector calculated using a pseudo feature amount extracted from the pseudo gait data and a pseudo response variable generated using the covariance matrix relating to the pseudo feature amount vector; and
processing of outputting a dataset including the measurement dataset vector and the pseudo dataset vector.

15. The estimation system according to claim 9, wherein
the estimation model is constructed by machine learning, and
the third processor is configured to execute the instructions to
display information that supports the user for making decision about taking an action.
